SEQUENCE LISTING (23 sequences)

SEQ ID NO: 1 - RNA, 30 nt, Artificial Sequence
  Synthetic Polynucleotide
guuuuagagcuagaaauagcaaguuaaaau

SEQ ID NO: 2 - RNA, 40 nt, N. meningitidis
guuguagcucccuuucucgaaagagaaccguugcuacaau

SEQ ID NO: 3 - RNA, 44 nt, N. meningitidis
guuguagcucccuuucucgaaagagaaccguugcuacaauaagg

SEQ ID NO: 4 - RNA, 101 nt, N. meningitidis
guuguagcucccuuucucgaaagagaaccguugcuacaauaaggccgucugaaaagaugugccgcaacgcucugccccuuaaagcuucugcuuuaacgggc

SEQ ID NO: 5 - DNA, 45 bp, Artificial Sequence
  Synthetic Polynucleotide
ctttggaagaacaacttaaggtcagattattttgcttagtaaact

SEQ ID NO: 6 - DNA, 45 bp, Artificial Sequence
  Synthetic Polynucleotide
agcagctgaaacagtgcagagtaagatttttatatgatgccttta

SEQ ID NO: 7 - RNA, 34 nt, Artificial Sequence
  Synthetic Polynucleotide
cuaauuccucucuucuccucucuccagguuguag

SEQ ID NO: 8 - RNA, 36 nt, S. pyogenes
guuuuagagcuaugcuguuuugaauggucccaaaac

SEQ ID NO: 9 - RNA, 38 nt, S. pyogenes
uuguuggaaccauucaaaacagcauagcaaguuaaaau

SEQ ID NO: 10 - RNA, 36 nt, N. meningitidis
guuguagcucccuuucucauuucgcagugcuacaau

SEQ ID NO: 11 - RNA, 36 nt, N. meningitidis
auugucgcacugcgaaaugagaaccguugcuacaau

SEQ ID NO: 12 - RNA, 36 nt, S. thermophilus
guuuuuguacucucaagauuuaaguaacuguacaac

SEQ ID NO: 13 - RNA, 37 nt, S. thermophilus
cuuacacaguuacuuaaaucuugcagaagcuacaaag

SEQ ID NO: 14 - RNA, 36 nt, T. denticola
guuugagaguuguguaauuuaagauggaucucaaac

SEQ ID NO: 15 - RNA, 38 nt, T. denticola
auuuaagauccaucuuaaauuacacaacgaguucaaau

SEQ ID NO: 16 - RNA, 18 nt, Artificial Sequence
  Synthetic Polynucleotide
guuguagcucccuuucuc

SEQ ID NO: 17 - RNA, 18 nt, Artificial Sequence
  Synthetic Polynucleotide
gagaaccguugcuacaau

SEQ ID NO: 18 - RNA, 22 nt, Artificial Sequence
  Synthetic Polynucleotide
nsnunbnbnbbnbbnnnhagvh

SEQ ID NO: 19 - DNA, 7113 bp, Artificial Sequence
  Synthetic Polynucleotide
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggccgcccggtttatcgttaatatggatcaatttgaacagttgattaacgtgtctctgctcaagtctttgatcaaaacgcaaatcgacgaaaatgtgtcggacaatatcaagtcgatgagcgaaaaactaaaaaggctagaatacgacaatctcacagacagcgttgagatatacggtattcacgacagcaggctgaataataaaaaaattagaaactattatttaaccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaacttttaaacattctctcttttacaaaaataaacttattttgtactttaaaaacagtcatgttgtattataaaataagtaattagcttaacttatacataatagaaacaaattatacttattagtcagtcagaaacaactttggcacatatcaatattatgctctcgacaaataacttttttgcattttttgcacgatgcatttgcctttcgccttattttagaggggcagtaagtacagtaagtacgttttttcattactggctcttcagtactgtcatctgatgtaccaggcacttcatttggcaaaatattagagatattatcgcgcaaatatctcttcaaagtaggagcttctaaacgcttacgcataaacgatgacgtcaggctcatgtaaaggtttctcataaattttttgcgactttgaaccttttctcccttgctactgacattatggctgtatataataaaagaatttatgcaggcaatgtttatcattccgtacaataatgccataggccacctattcgtcttcctactgcaggtcatcacagaacacatttggtctagcgtgtccactccgcctttagtttgattataatacataaccatttgcggtttaccggtactttcgttgatagaagcatcctcatcacaagatgataataagtataccatcttagctggcttcggtttatatgagacgagagtaaggggtccgtcaaaacaaaacatcgatgttcccactggcctggagcgactgtttttcagtacttccggtatctcgcgtttgtttgatcgcacggttcccacaatggttaattcgagctcgcccaaaccgggcgcgcctaattcctctcttctcctctctccaggttgtagctccctttctcgaaagagaaccgttgctacaataaggccgtctgaaaagatgtgccgcaacgctctgccccttaaagcttctgctttaacgggcaataaaatatctttattttcattacatctgtgtgttggttttttgtgtgggatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgggctgcaggtcgaggcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgatctgacaatgttcagtgcagagactcggctacgcctcgtggactttgaagttgaccaacaatgtttattcttacctctaatagtcctctgtggcaaggtcaagattctgttagaagccaatgaagaacctggttgttcaataacattttgttcgtctaatatttcactaccgcttgacgttggctgcacttcatgtacctcatctataaacgcttcttctgtatcgctctggacgtcatcttcacttacgtgatctgatatttcactgtcagaatcctcaccaacaagctcgtcatcgctttgcagaagagcagagaggatatgctcatcgtctaaagaactacccattttattatatattagtcacgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaaaaaagatttgcgctttactcgacctaaactttaaacacgtcatagaatcttcgtttgacaaaaaccacattgtggccaagctgtgtgacgcgacgcgcgctaaagaatggcaaaccaagtcgcgcgagcgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 20 - DNA, 8387 bp, Artificial Sequence
  Synthetic Polynucleotide
ggcctaactggccggtacctgagctcgctagcctcgaggatatcaagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgggggtgaggctccggtgcccgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaacaccgccaccatggtgcctaagaagaagagaaaggtggctgccttcaaacctaattcaatcaactacatcctcggcctcgatatcggcatcgcatccgtcggctgggcgatggtagaaattgacgaagaagaaaaccccatccgcctgattgatttgggcgtgcgcgtatttgagcgtgccgaagtaccgaaaacaggcgactcccttgccatggcaaggcgtttggcgcgcagtgttcgccgcctgacccgccgtcgcgcccaccgcctgcttcggacccgccgcctattgaaacgcgaaggcgtattacaagccgccaattttgacgaaaacggcttgattaaatccttaccgaatacaccatggcaacttcgcgcagccgcattagaccgcaaactgacgcctttagagtggtcggcagtcttgttgcatttaatcaaacatcgcggctatttatcgcaacggaaaaacgagggcgaaactgccgataaggagcttggcgctttgcttaaaggcgtagccggcaatgcccatgccttacagacaggcgatttccgcacaccggccgaattggctttaaataaatttgagaaagaaagcggccatatccgcaatcagcgcagcgattattcgcatacgttcagccgcaaagatttacaggcggagctgattttgctgtttgaaaaacaaaaagaatttggcaatccgcatgtttcaggcggccttaaagaaggtattgaaaccctactgatgacgcaacgccctgccctgtccggcgatgccgttcaaaaaatgttggggcattgcaccttcgaaccggcagagccgaaagccgctaaaaacacctacacagccgaacgtttcatctggctgaccaagctgaacaacctgcgtattttagagcaaggcagcgagcggccattgaccgataccgaacgcgccacgcttatggacgagccatacagaaaatccaaactgacttacgcacaagcccgtaagctgctgggtttagaagataccgcctttttcaaaggcttgcgctatggtaaagacaatgccgaagcctcaacattgatggaaatgaaggcctaccatgccatcagccgtgcactggaaaaagaaggattgaaagacaaaaaatccccattaaacctttctcccgaattacaagacgaaatcggcacggcattctccctgttcaaaaccgatgaagacattacaggccgtctgaaagaccgtatacagcccgaaatcttagaagcgctgttgaaacacatcagcttcgataagttcgtccaaatttccttgaaagcattgcgccgaattgtgcctctaatggaacaaggcaaacgttacgatgaagcctgcgccgaaatctacggagaccattacggcaagaagaatacggaagaaaagatttatctgccgccgattcccgccgacgaaatccgcaaccccgtcgtcttgcgcgccttatctcaagcacgtaaggtcattaacggcgtggtacgccgttacggctccccagctcgtatccatattgaaactgcaagggaagtaggtaaatcgtttaaagaccgcaaagaaattgagaaacgccaagaagaaaaccgcaaagaccgggaaaaagccgccgccaaattccgagagtatttccccaattttgtcggagaacccaaatccaaagatattctgaaactgcgcctgtacgagcaacaacacggcaaatgcctgtattcgggcaaagaaatcaacttaggccgtctgaacgaaaaaggctatgtcgaaatcgaccatgccctgccgttctcgcgcacatgggacgacagtttcaacaataaagtactggtattgggcagcgaaaaccaaaacaaaggcaatcaaaccccttacgaatacttcaacggcaaagacaacagccgcgaatggcaggaatttaaagcgcgtgtcgaaaccagccgtttcccgcgcagtaaaaaacaacggattctgctgcaaaaattcgatgaagacggctttaaagaacgcaatctgaacgacacgcgctacgtcaaccgtttcctgtgtcaatttgttgccgaccgtatgcggctgacaggtaaaggcaagaaacgtgtctttgcatccaacggacaaattaccaatctgttgcgcggcttttggggattgcgcaaagtgcgtgcggaaaacgaccgccatcacgccttggacgccgtcgtcgttgcctgctcgaccgttgccatgcagcagaaaattacccgttttgtacgctataaagagatgaacgcgtttgacggtaaaaccatagacaaagaaacaggagaagtgctgcatcaaaaaacacacttcccacaaccttgggaatttttcgcacaagaagtcatgattcgcgtcttcggcaaaccggacggcaaacccgaattcgaagaagccgataccctagaaaaactgcgcacgttgcttgccgaaaaattatcatctcgccccgaagccgtacacgaatacgttacgccactgtttgtttcacgcgcgcccaatcggaagatgagcgggcaagggcatatggagaccgtcaaatccgccaaacgactggacgaaggcgtcagcgtgttgcgcgtaccgctgacacagttaaaactgaaagacttggaaaaaatggtcaatcgggagcgcgaacctaagctatacgaagcactgaaagcacggctggaagcacataaagacgatcctgccaaagcctttgccgagccgttttacaaatacgataaagcaggcaaccgcacccaacaggtaaaagccgtacgcgtagagcaagtacagaaaaccggcgtatgggtgcgcaaccataacggtattgccgacaacgcaaccatggtgcgcgtagatgtgtttgagaaaggcgacaagtattatctggtaccgatttacagttggcaggtagcgaaagggattttgccggatagggctgttgtacaaggaaaagatgaagaagattggcaacttattgatgatagtttcaactttaaattctcattacaccctaatgatttagtcgaggttataacaaaaaaagctagaatgtttggttactttgccagctgccatcgaggcacaggtaatatcaatatacgcattcatgatcttgatcataaaattggcaaaaatggaatactggaaggtatcggcgtcaaaaccgccctttcattccaaaaataccaaattgacgaactgggcaaagaaatcagaccatgccgtctgaaaaaacgcccgcctgtccgttacccatacgatgttccagattacgctgcagctccagcagcgaagaaaaagaagctggattaactcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcagggatccgtttgcgtattgggcgctcttccgctgatctgcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgattcttctgacactagcgccaccatgaccgagtacaagcctaccgtgcgcctggccactcgcgatgatgtgccccgcgccgtccgcactctggccgccgctttcgccgactaccccgctacccggcacaccgtggaccccgaccggcacatcgagcgtgtgacagagttgcaggagctgttcctgacccgcgtcgggctggacatcggcaaggtgtgggtagccgacgacggcgcggccgtggccgtgtggactacccccgagagcgttgaggccggcgccgtgttcgccgagatcggcccccgaatggccgagctgagcggcagccgcctggccgcccagcagcaaatggagggcctgcttgccccccatcgtcccaaggagcctgcctggtttctggccactgtaggagtgagccccgaccaccagggcaagggcttgggcagcgccgtcgtgttgcccggcgtagaggccgccgaacgcgccggtgtgcccgcctttctcgaaacaagcgcaccaagaaaccttccattctacgagcgcctgggcttcaccgtgaccgccgatgtcgaggtgcccgagggacctaggacctggtgtatgacacgaaaacctggcgcctaatgatctagaaccggtcatggccgcaataaaatatctttattttcattacatctgtgtgttggttttttgtgtgttcgaactagatgctgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 21 - protein, 1081 aa, Artificial Sequence
  Synthetic Polypeptide
AAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 22 - protein, 199 aa, Artificial Sequence
  Synthetic Polypeptide
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 23 - protein, 286 aa, Artificial Sequence
  Synthetic Polypeptide
WHKILSAGIEAIQRNREDMTAQSGTTYIVVIRSPKGDPGLAAIIGRSGREGAGSKDAIFWGAPLASRLLPGAVKDAEMWDILQQRSALTLLEGTLLKRLTTAMAVPMTTDREDNPIAENLEPEWRDLRTVHDGMNHLFATLEKPGGITTLLLNAATNDSMTIAASCLERVTMGDTLHKETVPSYEVLDNQSYHIRRGLQEQGADIRSLVAGCLLVKFTSMMPFREEPRFSELIKGSNLDLEIYGVRAGLQDEADKVKVLTEPHAFVPLCFAAFFPILAVRFHQISM

(19) United States
(12) Patent Application Publication
(10) Pub. No.: US 2015/0197759 A1
(43) Pub. Date: Jul. 16, 2015

(54) MUTAGENESIS METHODS

(71) Applicant: LAM Therapeutics, Inc., Guilford, CT (US)

(72) Inventors: Tian Xu, Guilford, CT (US); Jonathan M. Rothberg, Guilford, CT (US)

(73) Assignee: LAM Therapeutics, Inc., Guilford, CT (US)

(21) Appl. No.: 14/597,038

(22) Filed: Jan. 14, 2015

Related U.S. Application Data
(60) Provisional application No. 61/927,458, filed on Jan. 14, 2014.

(51) Int. Cl. C12N 15/79; C12N 15/113

(52) U.S. Cl. CPC C12N 15/79; C12N 15/1137; C12N 2320/50

(57) ABSTRACT

In some embodiments, aspects of the disclosure provide methods and compositions that are useful for modifying (e.g., mutating) one or more alleles of a genomic locus within a cell. In some embodiments, methods and compositions described herein involve producing a chimeric spliced RNA molecule that includes a transcribed exon spliced to a nuclease interacting RNA segment. In some embodiments, the chimeric spliced RNA guides a DNA modifying enzyme (e.g., a nuclease) to a genomic locus in a cell resulting in modification of the locus.

### RELATED APPLICATIONS

This application claims priority under 35 U.S.C. §119 from U.S. provisional application Ser. No. 61/927,458, filed Jan. 14, 2014, the entirety of the contents is incorporated herein.

### BACKGROUND OF INVENTION

RNA-guided nucleases (e.g., Cas9) can be targeted to specific genomic target sites of interest using site-specific guide RNAs. A site-specific guide RNA can be designed to include both i) a targeting segment that is complementary to one strand of a genomic target site of interest and ii) a nuclease interacting segment that interacts with an RNA-guided nuclease. In use, the targeting segment of the guide RNA binds to a complementary sequence at the target genomic site, and the nuclease interacting segment of the guide RNA recruits the RNA-guided nuclease to the genomic target site resulting in targeted nucleic acid cleavage (e.g., double-stranded cleavage) at that site. In many cells, cleavage of a genomic site is repaired via intracellular repair mechanisms that can introduce mutations at the cleavage site. Therefore, RNA-guided nucleases can be used to introduce genomic mutations at known sites of interest.

### SUMMARY OF INVENTION

Current systems that use RNA-guided nucleases to produce genomic mutations are limited by the requirement that the target site be identified and incorporated into the guide RNA by design. In contrast, systems described herein are useful to introduce mutations into any expressed genomic site without designing a specific synthetic guide RNA for each genomic site or a DNA construct that encodes a specific synthetic guide RNA. Rather, systems described herein provide a nuclease interacting segment in a configuration that can be spliced onto an exon (downstream from the exon) that is transcribed from a genomic locus to produce a chimeric spliced RNA that can target a nuclease to the genomic locus. In some embodiments, an insertional nucleic acid construct that encodes a nuclease interacting RNA segment downstream from a splice acceptor site is integrated into a gene (e.g., an intron of a gene). As a result, transcription of the gene, followed by splicing of the transcribed RNA, produces a chimeric spliced RNA that includes at least one exon of the gene spliced to the nuclease interacting RNA segment. This chimeric spliced RNA can i) target one or more alleles of the corresponding genomic locus (via base paring between the one or more exons of the chimeric spliced RNA and the corresponding complementary strand of the genomic locus) and ii) recruit an RNA-guided nuclease to the one or more alleles (via interaction with the nuclease interacting segment of the chimeric spliced RNA), thereby promoting nuclease-based cleavage at the one or more alleles of the genomic locus. In some embodiments, the RNA-guided nuclease cleaves the genomic locus at or near the 3′ end of the exon that is targeted by the chimeric spliced RNA molecule (the RNA-guided nuclease is guided to that position by the chimeric spliced RNA molecule that is bound to the exon via complementary base pairing with the targeting portion of the chimeric spliced RNA molecule). It should be appreciated that the chimeric spliced RNA molecule can bind to the corresponding exon on each allele (e.g., both alleles in a diploid cell) of a genomic locus in a cell. Therefore, each allele of an expressed genomic locus can be targeted at the same position by the RNA-guided nuclease, and, as a result, a mutation can be introduced at the same position in each allele of an expressed genomic locus. Accordingly, it should be appreciated that two or more alleles (e.g., 3, 4, 5, 6, or more alleles of a multiploid cell) can be mutated as described herein.

In some embodiments, compositions and methods described herein can be used to produce mutations in both alleles of a plurality of genetic loci that are expressed, wherein each locus produces a transcript having a splice donor site, and wherein expression occurs within a host cell that is capable of RNA splicing. For example, compositions and methods described herein are useful in host cells that are eukaryotic. In some embodiments, host cells are in vitro. In some embodiments, host cells are in vivo. In some embodiments, host cells are cells in an organism, e.g., a mammal such as a mouse, non-human primate or human. Non-limiting examples of eukaryotic host cells include mammalian, avian, insect, yeast, plant and other eukaryotic host cells. In some embodiments, a host cell is a human host cell. Non-limiting examples of host cells include, without limitation stem cells, epithelial cells, endothelial cells, etc. In some embodiments, a host cell is a human stem cell.

Compositions and methods described herein can be used to generate a library of host cells having mutations at each of a plurality of different expressed genomic loci. Libraries may be produced by delivering (e.g., by transfection) insertional nucleic acid constructs of the disclosure to host cells and then isolating cells containing DNA into which one or more nucleic acid constructs have been inserted. Host cells can be produced having different numbers of mutations by adjusting the ratio of insertional nucleic acid constructs that are mixed with the cells during a transfection procedure. In some embodiments, each mutant cell in the library has on average a mutation at only one genomic locus at one or both alleles of a diploid cell (or multiple alleles in a cell of higher ploidy, e.g., a ploidy of 3n, 4n, 5n, 6n, 7n, 8n, etc.). It should be appreciated that the mutation introduced in each allele may be different when both alleles of a diploid cell undergo DNA break repair. However, in some embodiments each mutant cell in the library of diploid cells has on average a mutation at two or more different genomic loci at one or both alleles. In some embodiments, each mutant cell in a library of diploid cells has on average a mutation at both alleles of a single genomic locus. It also should be appreciated that different mutations can be produced at a given genomic locus and may be present in different host cells in a library. For example, an insertional construct described herein can integrate into different positions (e.g., introns) of an expressed genomic locus and consequently generate mutations in different exons (for example, at the 3′ end each different exon) of a genomics locus. In some embodiments, libraries are produced having many different cells each having a different integration site. In some embodiments, libraries are produced having a number of cells in a range of up to 10³, 10² to 10⁴, 10² to 10⁵, 10² to 10⁶, 10² to 10⁷, 10² to 10⁹, 10³ to 10⁶, 10³ to 10⁷, 10⁴ to 10⁶, 10⁴ to 10⁷, or 10⁴ to 10⁸, each cell having a different integration sites. In some embodiments, libraries can be constructed and arranged to contain different classes of genes by selecting out cells having insertions (random or target) within the particular classes of genes. For example, cells of a library may have insertions within genes encoding regulatory factors, metabolic factors, developmental factors, receptors (e.g., immune checkpoint receptors, G-protein coupled receptors), enzymes (e.g., kinases, phosphatases), transcription factors, structural proteins, motor proteins and other classes of genes, including genes encoding regulatory RNAs, such as miRNAs, non-coding RNAs (e.g., lncRNAs), etc.

In some embodiments, a library of genomic mutations can be screened to identify one or more loci that are sensitive to treatment with one or more candidate compounds. However, it should be appreciated that a library of mutations can be screened using any assay to identify one or more loci associated with a phenotype or property of interest.

Aspects of the invention relate to methods of producing, in a cells capable of splicing, such as a eukaryotic cell, a target-specific RNA molecule capable of guiding a DNA nuclease to a genomic target. In some embodiments, the methods comprise introducing a recombinant nucleic acid into a eukaryotic cell, wherein the recombinant nucleic acid comprises a first nucleic acid region that encodes a splice acceptor site upstream of a second nucleic acid region that encodes an RNA segment capable of interacting with an RNA-guided DNA nuclease. In some embodiments, the methods comprise integrating a recombinant nucleic acid into a genomic locus of a eukaryotic cell, wherein the recombinant nucleic acid comprises a first nucleic acid region that encodes a splice acceptor site upstream of a second nucleic acid region that encodes an RNA segment capable of interacting with an RNA-guided DNA nuclease.

Some aspects of the invention provide methods of promoting RNA-guided cleavage of a genomic DNA within a cell. In some embodiments, the methods comprise producing, in a cell, an RNA molecule that comprises a first RNA segment spliced to a second RNA segment, wherein the first RNA segment comprises an exonic sequence transcribed from a genomic locus and the second RNA segment comprises an RNA segment capable of interacting with an RNA-guided DNA nuclease. In some embodiments, the methods further comprise expressing, in the cell, an RNA-guided DNA nuclease.

Aspects of the invention relate to methods of producing, in a eukaryotic cell, a target specific nucleic acid that guides a DNA modifying enzyme. In some embodiments, the methods comprise introducing a recombinant nucleic acid into a eukaryotic cell, wherein the recombinant nucleic acid comprises a first nucleic acid region that encodes a splice acceptor site upstream of a second nucleic acid region that encodes an RNA segment capable of interacting with the DNA modifying enzyme. In some embodiments, the DNA modifying enzyme is an RNA-guided DNA nuclease. In some embodiments, the eukaryotic cell is a stem cell.

Aspects of the invention relate to a nucleic acid comprising a first nucleic acid region that encodes a splice acceptor site upstream of a second nucleic acid region that encodes an RNA segment capable of interacting with a DNA modifying enzyme. In some embodiments, the DNA modifying enzyme is an RNA-guided DNA nuclease.

In some embodiments, the recombinant nucleic acid is a DNA molecule. In some embodiments, the recombinant nucleic acid comprises transposon terminal sequences (e.g., at the 5′ end and 3′ ends of a linear recombinant nucleic acid). In some embodiments, the transposon terminal sequences comprise inverted terminal repeat sequences (ITRs). In some embodiments, the transposon terminal sequences comprise direct terminal repeat sequences. In some embodiments, the direct terminal repeat sequences flank the ITRs. In some embodiments, the transposon terminal sequences comprise a 5′ terminal CCY and a 3′ terminal GGG. In some embodiments, the transposon terminal sequences comprise a 5′ terminal CCC and a 3′ terminal GGG. In some embodiments, the transposon terminal sequences target TTAA insertion sites. In some embodiments, the transposon terminal sequences comprise PiggyBac transposon-specific inverted terminal repeat sequences (ITRs). In some embodiments, the transposon terminal sequences comprise Tagalong transposon-specific inverted terminal repeat sequences (ITRs). In some embodiments, the recombinant nucleic acid further comprises a third nucleic acid region encoding a selection or screening marker. In some embodiments, the selection or screening marker is an antibiotic resistance protein or a fluorescent or bioluminescent protein.

In some embodiments, the splice acceptor site comprises a sequence set forth as 5′-X₁X₂X₃-3′, wherein: X₁ is A; X₂ is G or C; and X₃ is A, G, C, or U, wherein a 3′ splice junction is between X₂ and X₃. In some embodiments, X₂ is G. In some embodiments, X₃ is A, G or C. In some embodiments, the splice acceptor site comprises a sequence set forth as 5′-X₁X₂X₃X₄X₅-3′, wherein: X₁ is A, C or U; X₂ is A; X₃ is G; X₄ is A, G or C; and X₅ is A, U or C, wherein a 3′ splice junction is between X₃ and X₄. In some embodiments, the splice acceptor site comprises a sequence set forth as 5′-X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂-3′ (SEQ ID NO: 18), wherein: X₁, X₃, X₅, X₇, X₉, X₁₂, X₁₅, X₁₆, and X₁₇ are each independently selected from A, G, C, and U; X₂ is C or G; X₄ is U; X₆, X₈, X₁₀,X₁₁, X₁₃, X₁₄ are each independently selected from G, C, and U; X₁₈ is A, C or U; X₁₉ is A; X₂₀ is G; X₂₁ is A, C, or G; and X₂₂ is A, U or C, wherein a 3′ splice site is between X₂₀ and X₂₁.

In some embodiments, the nuclease interacting segment comprises at least one stem portion that interacts with the RNA-guided DNA nuclease. In some embodiments, the nuclease interacting segment comprises first and second stem portions that are separated by non-complementary RNA nucleotides. In some embodiments, the first stem portion comprises a strand having a nucleotide sequence set forth as 5′-GUUGUAGC-3′. In some embodiments, the second stem portion comprises a nucleotide sequence set forth as 5′-UUCUC-3′. In some embodiments, complementary base pairs of the two strands of the second stem portion are covalently linked through a loop structure. In some embodiments, the nuclease interacting segment comprises a sequence set forth as 5′-GUUUUAGAGCUAGAAAUAGCAAGUUAAAAU-3′(SEQ ID NO: 1).

In some embodiments, the eukaryotic cell is a mammalian cell. In some embodiments, the eukaryotic cell is a plant cell. In some embodiments, the mammalian cell is a human cell.

In some embodiments, a recombinant nucleic acid encodes the RNA-guided DNA nuclease. In some embodiments, the RNA-guided DNA nuclease is a CRISPR-associated (Cas) nuclease. In some embodiments, the Cas nuclease is a Type II Cas nuclease. In some embodiments, the Cas nuclease is a Cas9 nuclease. In some embodiments, the Cas9 nuclease is a Neisseria meningitidis Cas9 nuclease. In some embodiments, the Cas9 nuclease is a Streptococcus thermophiles Cas9 nuclease. In some embodiments, the RNA-guided DNA nuclease introduces single-stranded breaks in DNA. In some embodiments, the RNA-guided DNA nuclease introduces double-stranded breaks in DNA. In some embodiments, the RNA-guided DNA nuclease is expressed under conditions that promote i) interaction between the RNA-guided DNA nuclease and the second RNA segment of the RNA molecule, and ii) DNA cleavage at one or more genomic loci encoding the exonic sequence. In some embodiments, DNA cleavage occurs within 5 base pairs upstream of a splice donor site of the exonic sequence.

In some embodiments, the one or more genomic loci are two or more alleles encoding the exonic sequence. In some embodiments, the two or more alleles are two alleles in a mammalian cell.

These and other aspects are described in more detail herein.

### BRIEF DESCRIPTION OF DRAWINGS

FIGS. A and 1B illustrate non-limiting embodiments of the generation of a chimeric spliced RNA molecule (containing Exon a′);

FIGS. 2A and 2B illustrate non-limiting embodiments of a nucleic acid cleavage system guided by a chimeric spliced RNA molecule (containing Exon a′) targeting multiple alleles, and DNA repair induced mutagenesis following targeted nucleic acid cleavage;

FIG. 3 illustrates a non-limiting embodiment transposon excision following DNA repair induced mutagenesis;

FIG. 4A-C illustrate non-limiting embodiments of nuclease interacting segments comprising sequences of targeting CRISPR associated RNA (crRNA) and transactivating crRNA (tracrRNA) from Neisseria meningitidis. SEQ ID NO: 2 is listed in FIG. 4A; SEQ ID NO: 3 is listed in FIG. 4B; SEQ ID NO: 4 is listed in FIG. 4C;

FIG. 4D illustrates a type II CRISPR system utilizing an insertional recombinant nucleic acid comprising a nuclease interacting segment comprising sequences of targeting crRNA and tracrRNA from Neisseria meningitides, and flanked by PiggyBac transposon sequences;

FIG. 4E illustrates two exon/intron boundaries of the human Dystrophin gene Exon 13 is SEQ ID NO: 5 and Exon 24 is SEQ ID NO: 6;

FIG. 5A illustrates a non-limiting embodiments of consensus splice donor and acceptor sites;

FIG. 5B illustrates a non-limiting embodiment of a chimeric RNA. SEQ ID NO: 7 is listed in FIG. 5B;

FIG. 6A illustrates a non-limiting embodiment of a nucleic acid construct containing an exon fitted with a protospacer adjacent motif (PAM) and a portion encoding an RNA comprising a splice acceptor and nuclease interacting segment;

FIG. 6B illustrates a non-limiting embodiment of a nucleic acid construct encoding an RNA-guided nuclease;

FIG. 7 illustrates a non-limiting embodiment of a work flow for evaluating CRISPR activity;

FIG. 8 illustrates a non-limiting embodiment of a system for targeting a modified nuclease to a genomic site, where Exon a′ denotes the spliced RNA molecule;

FIG. 9 provides a non-limiting example of a sequence (SEQ ID NO: 19) of an insertional recombinant nucleic acid. The recombinant nucleic acid comprises a splice acceptor site upstream of a nucleic acid region that encodes an RNA segment capable of interacting with a RNA-guided nuclease; and

FIG. 10 provides a non-limiting example of a sequence of a nucleic acid engineered to express a Cas9 nuclease. The DNA sequence corresponds to SEQ ID NO: 20, and the protein sequences, from left to right, correspond to SEQ ID NOs: 21, 22, and 23.

### DETAILED DESCRIPTION OF INVENTION

In some embodiments, aspects of the disclosure provide methods and compositions that are useful for modifying (e.g., mutating) one or more alleles of a genomic locus within a cell. In some embodiments, methods and compositions described herein involve producing a chimeric spliced RNA molecule that includes a transcribed exon spliced to a nuclease interacting RNA segment.

Aspects of the disclosure relate to methods and compositions for modifying target nucleic acids intracellularly. In some embodiments, a target nucleic acid is modified intracellularly by a nuclease that is guided to the target nucleic acid by a chimeric spliced RNA molecule that includes a first targeting segment that is complementary to the target nucleic acid (e.g., to one strand of a double stranded DNA molecule at the target site) and that is spliced to a second segment that is capable of interacting with the nuclease. In some embodiments, the first segment includes at least one exon, and the second segment includes an RNA capable of interacting with a CRISPR-associated nuclease (e.g., a Cas9 nuclease).

In some embodiments, the chimeric spliced RNA molecule is produced intracellularly and includes an RNA segment corresponding to a transcribed genomic region (e.g., including one or more exons) spliced to a recombinant RNA segment, wherein the recombinant RNA segment is encoded on a recombinant nucleic acid that is integrated into an intron of the transcribed genomic region. Accordingly, in some embodiments aspects of the disclosure relate to providing, within a cell, an RNA that contains a splice acceptor site connected to an RNA capable of interacting with a nuclease. In some embodiments, the RNA is provided by integrating a construct into a genomic site.

In some embodiments, the chimeric spliced RNA molecule binds to the expressed genomic locus (e.g., via complementary base-pairing between the targeting segment and the complementary strand of the genomic DNA at the expressed locus) and a nuclease that binds to the nuclease interacting segment of the chimeric spliced RNA molecule. As a result, the nuclease is guided to the genomic locus. In some embodiments, the nuclease cleaves the genomic DNA (e.g., on one or both strands) at or near the genomic site having a sequence that is complementary to the targeting segment on the chimeric spliced RNA. In some embodiments, a host cell repair mechanism repairs the cleaved DNA and introduces a mutation at the cleavage site during the repair process. It should be appreciated that this process can be targeted to multiple alleles of an expressed genomic locus (e.g., both alleles in a diploid organism), even though the recombinant nucleic acid that encodes the nuclease interacting segement is integrated into only one allele of the genomic locus. Accordingly, methods and compositions described herein can be used to target nuclease activity to multiple alleles of a locus in a cell (e.g., two alleles in a diploid cell). In some embodiments, the nuclease introduces double strand breaks in the one or more alleles.

In some embodiments, aspects of the disclosure are useful to produce host cells having one or more modifications (e.g., mutations) at expressed genomic loci (e.g., at two or more alleles of each expressed genomic locus that is targeted). In some embodiments, libraries of host cells can be produced with mutations in different genetic loci and these libraries can be screened to identify one or more loci of interest (e.g., associated with a disease or a response to therapy or other property of interest).

In some embodiments, a host cell can be a cell that has one or more mutations that increases the frequency of errors during repair and thereby increases the frequency of mutations generated in a process described herein.

Recombinant nucleic acids disclosed herein can be delivered in any suitable vector. For example, a recombinant nucleic acid can have sequences at either end that promote recombination or that target an insertion site of interest. In some embodiments, the recombinant nucleic acid can be delivered in a viral vector, such as, for example, a retrovirus (e.g., a lentivirus), a herpesvirus (e.g., herpes simplex virus type-1), etc.

In some embodiments, the recombinant nucleic acid is delivered in a transposon. In some embodiments, the recombinant nucleic acid is delivered in a vector that comprises TTAA-specific, short repeat elements of a transposon system. In some embodiments, the recombinant nucleic acid is delivered in a vector that comprises elements that exhibit a preference for TTAA target sites, and insert within an FP-locus or at other regions of a genome.

In some embodiments, the recombinant nucleic acid is delivered in a vector that comprises a PiggyBac (PB) transposon element, which is a mobile genetic element that efficiently transposes via a “cut and paste” mechanism. In some embodiments, during transposition, a PB transposase recognizes transposon-specific inverted terminal repeat sequences (ITRs) located on both ends of the transposon vector and efficiently moves the contents from the original sites and efficiently integrates them into a TTAA chromosomal site.

In some embodiments, a recombinant nucleic acid engineered to express an appropriate transposase (e.g., a Piggy Bac (PB) transposase, Sleeping Beauty (SB) transposase, Transposase Tn5, etc.) is delivered to host cells to bring about a desired type of transposition in the cells.

In some embodiments, the recombinant nucleic acid is delivered in a vector that comprises sequences of a mobile host DNA insertion element within the few-polyhedra (FP) locus of the baculovirus AcMNPV or GmMNPV. In some embodiments, the recombinant nucleic acid is delivered in a vector that comprises transposon sequences of a tagalong (alternatively referred to as TFP3) transposon.

In some embodiments, the recombinant nucleic acid is delivered in a vector that comprises a LOOPER element, which has sequence homology to piggyBac. In some embodiments, the LOOPER element is a DNA element that terminates in 5′ CCY . . . GGG 3′, and targets TTAA insertion sites.

In some embodiments, the recombinant nucleic acid is delivered in a vector that comprises a TTAA-specific fossil repeat element, such as, for example, MER75 and MER85. In some embodiments, the TTAA-specific fossil repeat element terminates in 5′ CCC . . . GGG 3′, and targets TTAA insertion sites.

In some embodiments, the recombinant nucleic acid is delivered in a vector that comprises flanking transposon sequences of a Maize Ac/Ds system. In some embodiments, the recombinant nucleic acid is delivered in a vector that comprises a P element. In some embodiments, the recombinant nucleic acid is delivered in a vector that comprises sequences of bacterial transposons belonging to the Tn family. In some embodiments, the recombinant nucleic acid is delivered in a vector that comprises Alu sequences. In some embodiments, the recombinant nucleic acid is delivered in a vector that comprises a Mariner-like element. In some embodiments, the recombinant nucleic acid is delivered in a vector that comprises sequences that facilitate Mu phage transposition. In some embodiments, the recombinant nucleic acid is delivered in a vector that comprises transposon sequences from the retrotransposon family Ty1, Ty2, Ty3, Ty4 or Ty5. In some embodiments, the recombinant nucleic acid is delivered in a vector that comprises transposon sequences of a helitron. In some embodiments, the recombinant nucleic acid is delivered in a Sleeping Beauty transposon.

In some embodiments, the recombinant nucleic acid is delivered in a T-DNA vector (e.g., for delivery to plant cells).

It should be appreciated that the recombinant nucleic acid may be inserted into a genomic locus using any appropriate method. In some embodiments, an insertional recombinant nucleic acid may be engineered to contain flanking sequences of that are homologous to a genomic locus of interest (e.g., an oncogene or an integrated viral gene) to facilitate targeted insertion into a target genomic locus, e.g., through homologous recombination. In some embodiments, an insertional recombinant nucleic acid contains flanking sequences that are homologous to a genomic locus of interest, in which the flanking sequences are up to 100 bp, up to 500 bp, up to 1 kb, up to 2 kb, up to 3 kb, or up to 5 kb. In some embodiments, the flanking sequences are in a range of 10 by to 100 bp, 100 by to 500 bp, 100 by to 1 kb, 100 by to 2 kb, 500 by to 3 kb, or 1 kb to 5 kb.

In some embodiments, a recombinant nucleic acid that encodes a nuclease interacting segment of an RNA molecule downstream from a splice acceptor site is provided. When the recombinant nucleic acid is introduced into a host cell (e.g., via transfection, viral transduction, electroporation, or other technique) it can integrate within an expressed template nucleic acid downstream from a splice donor site of an exon of the expressed template nucleic acid (e.g., within an intron of an expressed region of a genomic nucleic acid). In some embodiments, the recombinant nucleic acid is delivered to a cell via transfection with or without a carrier (e.g., a lipid-based carrier) that facilitates transcription. In some embodiments, the recombinant nucleic acid is delivered to a cell via viral transduction.

The resulting transcript from this site can be spliced to produce a chimeric spliced RNA molecule that contains the upstream exon from the expressed nucleic acid spliced onto the nuclease interacting RNA segment. This chimeric spliced molecule can act as a targeting molecule to target a nuclease to the expressed template nucleic acid. The exon portion of the chimeric spliced RNA molecule acts as a targeting sequence—it is complementary to one strand of the expressed template nucleic acid and can bind by complementary base pairing. This targets the nuclease to that template nucleic acid (e.g., genomic nucleic acid) via the interacting RNA segment that recruits the nuclease to the site of the bound chimeric spliced RNA.

Accordingly, in some embodiments, aspects of the disclosure relate to compositions and methods of producing an RNA molecule that targets a nuclease to a particular target site or region on a nucleic acid. In some embodiments, a targeting RNA molecule contains both a targeting region and a nuclease interacting region. In some embodiments, the two regions are spliced together within a cell in order to produce the targeting RNA within the cell. In the presence of both the target nucleic acid and the nuclease, the targeting RNA acts as an agent that brings the target nucleic acid and the nuclease together thereby promoting cleavage of the target nucleic acid by the nuclease. The targeting segment of the targeting RNA corresponds to a portion transcribed from the target nucleic acid and is therefore complementary to one strand of the target nucleic acid (e.g., genomic DNA) and can bind to the target nucleic acid (e.g., via complementary base pairing with the target DNA). In some embodiments, the nuclease interacting segment of the targeting RNA interacts with the nuclease and thereby promotes cleavage of the target nucleic acid. However, it should be appreciated that in some embodiments a modified nuclease can be used. A modified nuclease can retain its ability to bind to the nuclease interacting segment of the targeting RNA, but be modified to remove it nucleic acid cleavage activity and/or to introduce one or more additional effector functions (e.g., regulatory and/or enzymatic as described in more detail herein).

Accordingly, in some embodiments a targeting RNA includes two regions: i) a region that is complementary to a nucleic acid target, and ii) a region that interacts with a nuclease. When provided in a cell along with the nuclease, the targeting RNA binds to the target nucleic acid (via its complementary first region) and promotes cleavage of the target nucleic acid by interacting with the nuclease (via the region that interacts with the nuclease).

In some embodiments, some aspects of the disclosure are illustrated with reference to FIGS. 1A and 1B. In particular, non-limiting embodiments of the generation of a targeted genomic DNA cleavage system are illustrated in FIGS. 1A and 1B. In FIG. 1A, a recombinant nucleic acid encoding an RNA comprising a nuclease interacting segment downstream of a splice acceptor (SA) site is provided. In some embodiments, a transcriptional termination sequence (stop) is encoded downstream of the nuclease interacting segment. In some embodiments, a polyadenylation signal is encoded downstream of the nuclease interacting segment.

Step 100A depicts an insertion of the recombinant nucleic acid into an intron of a genomic locus between two exons (Exon a and Exon b), downstream of the splice donor (SD) site of the first exon (Exon a). It should be appreciated that insertion of a recombinant nucleic acid may result from a random integration or a targeted integration into a site in the genome (e.g., a site within an intron). In the case of random or targeted integration, different cells having different integration sites can be isolated (e.g., randomly or using a selection or a screen) and further evaluated. It should also be appreciated that a recombinant nucleic acid can be integrated into any intron in a gene. Depending on the particular intron, the resulting difference would be that the cleavage (and subsequent error correction—if any) would be in a different allelic position, e.g., a different exon. It should also be appreciated that methods disclosed are not limited to instances in which insertion occurs within an intron. In some embodiments, insertion may occur within or adjacent to an intron, an exon, untranslated region or another position provided that the desired splicing is still effective.

In FIGS. 1A and 1B, the splice donor site of Exon a is separated from the splice acceptor site of the nuclease interacting segment by the portion of the intron leading up to the genomic insertion site of the recombinant nucleic acid. At step 101A, transcription from the promoter of the genomic locus produces an RNA transcript comprising Exon a′ with its splice donor site upstream from the splice acceptor site of the nuclease interacting segment. Splicing of the RNA transcript produces a spliced chimeric RNA molecule including the nuclease interacting segment immediately downstream of Exon a′, as shown in step 101A. FIG. 1A also illustrates, at step 101A, the splice by-product that results from the splicing reaction. The splice by-product includes the splice donor and acceptor sites and a portion of the intron.

In FIG. 1B, a recombinant nucleic acid encoding an RNA comprising a nuclease interacting segment downstream of a splice acceptor (SA) site is flanked by transposon terminal repeats (TR). The transposon terminal repeats promote the integration of the recombinant nucleic acid into the genome. In FIG. 1B, the transposon construct integrates into an intron between two exons (Exon a and Exon b) of a genomic locus as shown in steps 100B-101B. Similar to FIG. 1A, splicing of the RNA transcript produces a spliced chimeric RNA molecule including the nuclease interacting segment immediately downstream of Exon a, as shown in step 101B. FIG. 1B also illustrates, at step 101B, the splice by-product that results from the splicing reaction. The splice by-product includes the splice donor and acceptor sites, a portion of the intron, and the first transposon terminal repeat.

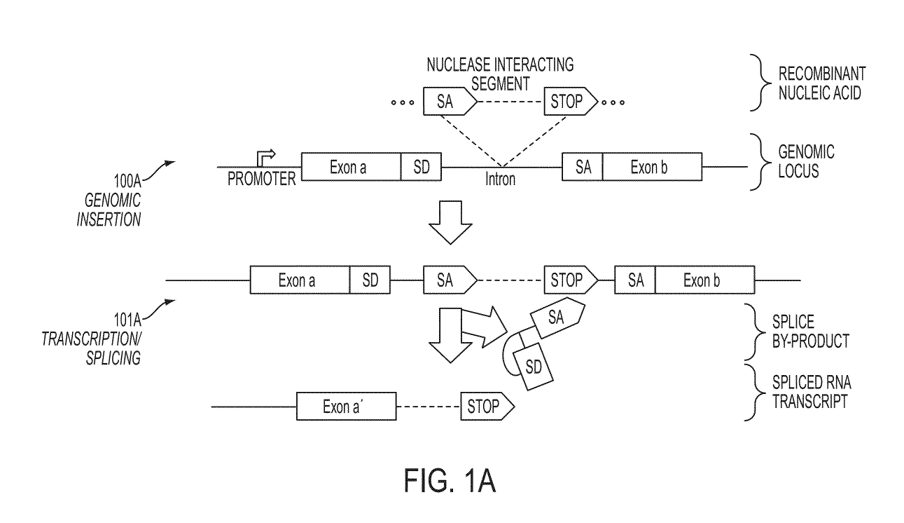

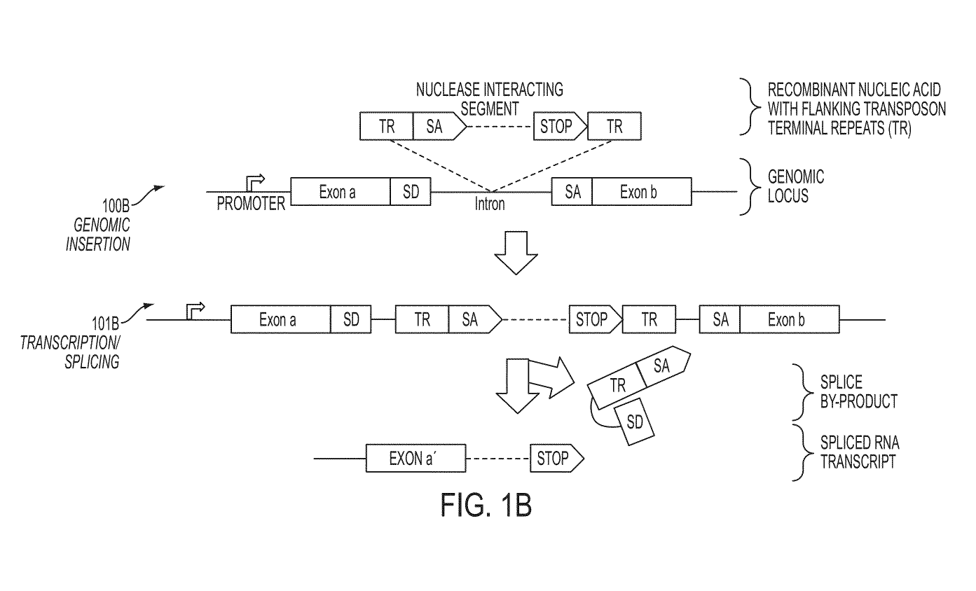

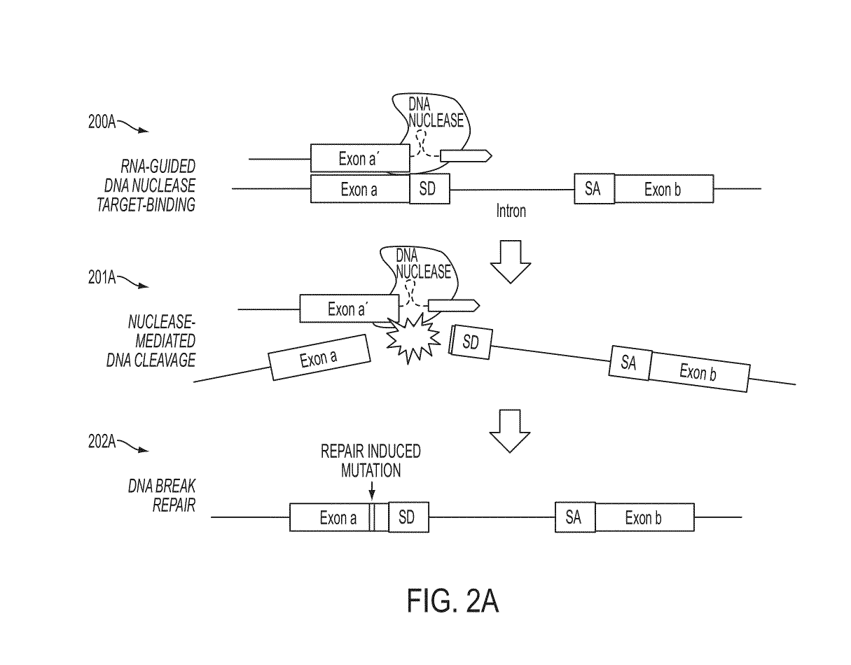

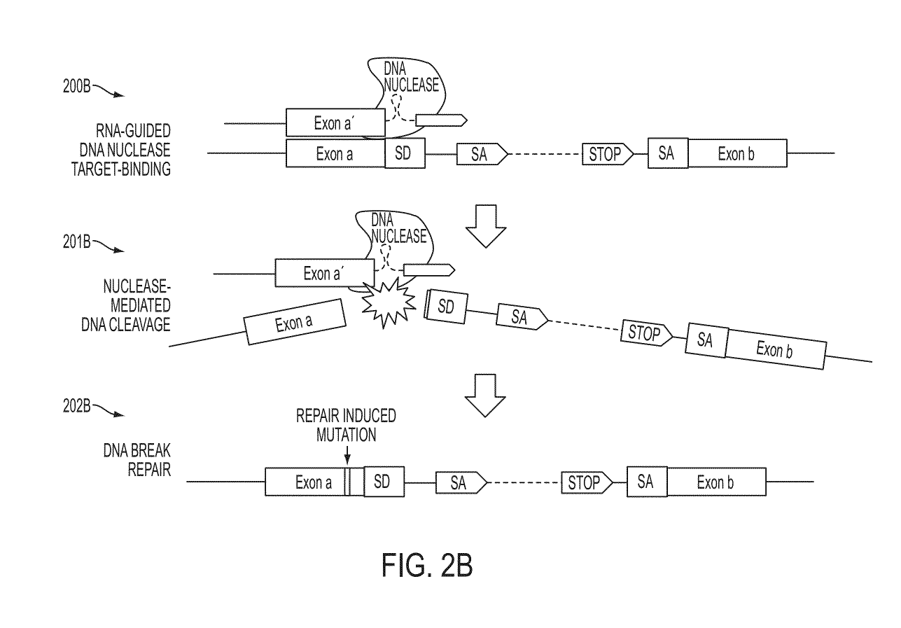

As described herein, multiple alleles of a genomic locus can be targeted by a chimeric spliced RNA molecule that is expressed from a single integrated nucleic acid. FIGS. 2A and 2B illustrate non-limiting embodiments of two alleles of a genomic locus being targeted by a chimeric spliced RNA molecule that is expressed from only one of the alleles in which the recombinant nucleic acid was integrated. It also should be appreciated that the process illustrated in FIGS. 2A and 2B can result in the production of a genomic mutation at multiple alleles (e.g., both alleles in a diploid organism) of a genetic locus within a cell.

As depicted in FIG. 2A, a chimeric spliced RNA molecule (e.g., as generated by the steps of FIGS. 1A or 1B) can promote RNA-guided DNA nuclease target-binding to an allele of the genomic locus (that does not contain the integrated nucleic acid in the intron), as illustrated in step 200A. At step 200A, the chimeric spliced RNA molecule (spliced RNA transcript) binds to the genomic locus that encodes Exon a (via base-pairing interaction between the Exon a segment on the spliced RNA and the complementary strand of Exon a at the genomic locus). The chimeric spliced RNA molecule bound to the genomic Exon a locus also recruits an RNA-guided DNA nuclease (via interaction between the nuclease and the nuclease interacting segment of the chimeric spliced RNA molecule) expressed in the same cell.

The nuclease that is recruited to the genomic site by the chimeric spliced RNA molecule can cleave the genomic nucleic acid as illustrated in step 201A.

The resulting cleaved genomic region can be repaired by intracellular repair enzymes. However, in some instances the repair process introduces a mutation at the cleavage site as illustrated in step 202A. Accordingly, the process illustrated in FIG. 2A can result in the production of a genomic mutation at the cleavage site.

As depicted n FIG. 2B, a chimeric spliced RNA molecule can promote RNA-guided DNA nuclease target-binding to another allele of the genomic locus (that contains the integrated nucleic acid in the intron), as illustrated in step 200B. The chimeric spliced RNA guides a DNA nuclease to the genomic locus of Exon a as shown in step 200B. The nuclease that is recruited to the genomic site can cleave the genomic nucleic acid as illustrated in step 201B. Subsequently, intracellular DNA repair enzymes can introduce a mutation at the break site during the repair process to produce a genomic locus with a repair-induced mutation in Exon a, as illustrated in step 202B.

Accordingly, as illustrated in FIGS. 2A and 2B, a mutation can be introduced into multiple alleles of the genomic locus via a cellular DNA repair process as described herein.

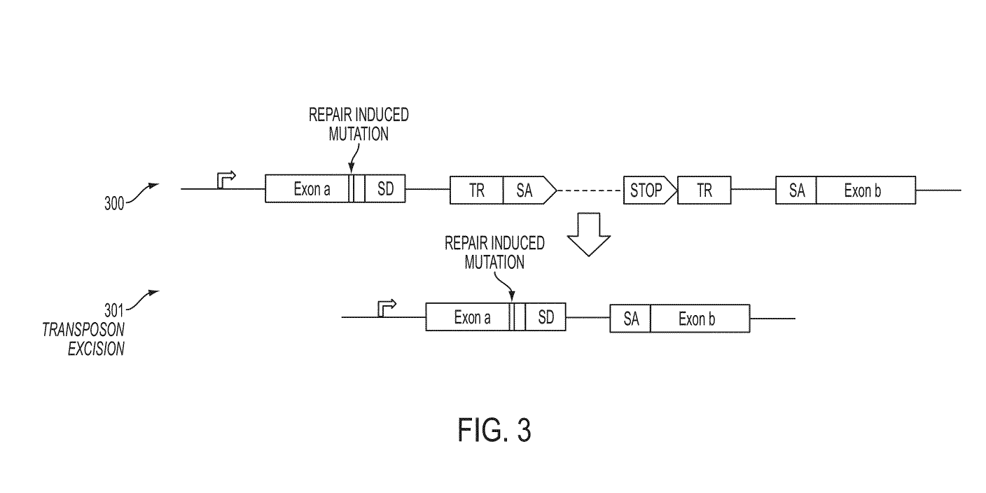

In some embodiments, the integrated recombinant nucleic acid (flanked by the transposon repeats) is excised (e.g., via a transposase-induced excision) thereby leaving the repair-induced mutation at the genomic locus of Exon a, but removing the recombinant nucleic acid (along with the nuclease interacting segment) from the genome, as illustrated in FIG. 3.

In some embodiments, a transcriptional termination sequence is located downstream from the nuclease interacting segment on the recombinant nucleic acid that is integrated into the host cell genome (the recombinant nucleic acid that encodes the nuclease interacting segment downstream from the splice acceptor site). This terminates transcription of the chimeric RNA within the sequence encoded by the recombinant nucleic acid and prevents transcription from continuing through to any further introns or exons downstream from the site of genomic integration.

In some embodiments, the recombinant nucleic acid that is inserted into the host genome does not include a promoter sequence upstream from the splice acceptor site.

In some embodiments, one or more transposon terminal repeat sequences (e.g., direct or indirect repeats, or a combination thereof) are present at both ends of the recombinant nucleic acid encoding the nuclease interacting segment downstream from the splice acceptor site. These transposon terminal repeat sequences can promote insertion of the recombinant nucleic acid into the genome of a host cell.

In some embodiments, one or more selectable markers (e.g., a drug resistance marker) are encoded on the recombinant nucleic acid encoding the nuclease interacting segment downstream from the splice acceptor site. The one or more selectable markers can be used to select for host cells in which the recombinant nucleic acid has integrated into the genome.

In some embodiments, one or more enzymes that promote transposon integration and/or excision (e.g., one or more transposases) are encoded on the recombinant nucleic acid that is integrated into the host cell genome. In some embodiments, one or more RNA-guided nucleases (e.g., Cas9) are encoded on the recombinant nucleic acid that is integrated into the host cell genome. However, it should be appreciated that the one or more enzymes that promote transposon integration and/or excision and/or one or more RNA-guided nucleases can be encoded on separate nucleic acids (e.g., other vectors, for example self-replicating vectors, or at one or more other genomic loci within a host cell).

### Nuclease Interacting Segments:

In some embodiments, the disclosure provides recombinant nucleic acids that encode RNA having nuclease interacting segments. In some embodiments, a nuclease interacting segment includes one or more sequences that can promote formation of a secondary structure that interacts with an RNA-guided nuclease. In some embodiments, a nuclease interacting segment includes one or more sequences that can promote formation of a substantially double stranded RNA structure (e.g., a stem) that interacts with an RNA-guided nuclease. In some embodiments, a nuclease interacting segment possesses characteristics of the natural structure of a crRNA:tracrRNA complex that interacts with RNA guided nucleases. In some embodiments, a nuclease interacting segment forms a stem that mimics a base-paired structure that forms between targeting crRNA and tracrRNA molecules in a Type II CRISPR system. In some embodiments, a stem of a nuclease interacting segment includes one or more based-paired structures having sequences shown in Table 1 or portions thereof. For example, in some embodiments a stem of a nuclease interacting segment includes at least 5 nucleotides (e.g., 5-10, 10-15, 15-20, or more nucleotides) of a base-paired structure shown in Table 1 or a portion thereof (e.g., of one stem or both stems of a base-paired structure or a portion thereof of Table 1). In some embodiment, a stem of a nuclease interacting segment includes at least 5 nucleotides (e.g., 5-10, 10-15, 15-20, or more nucleotides) that have a sequence that is 90%, 90-95%, around 95%, or 95-100% identical to a sequence of a base-paired structure shown in Table 1 or a portion thereof (e.g., of one stem or both stems of a base-paired structure or a portion thereof of Table 1).

TABLE 1
RNA-Guided Nuclease Interacting Regions

| | Base-paired structure between targeting crRNA (top |
|---|---|
| Species | strand)and activating tracrRNA molecules (bottom strand) |
| S. pyogenes | |
| SEQ ID NO: 8 SEQ ID NO: 9 | 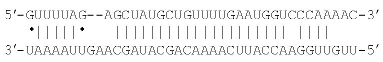 |
| N. meningitidis | |
| SEQ ID NO: 10 SEQ ID NO: 11 | 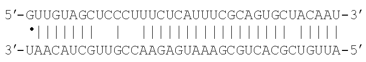 |
| S. thermophilus | |
| SEQ ID NO: 12 SEQ ID NO: 13 | 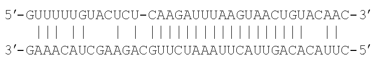 |
| T. denticola | |
| SEQ ID NO: 14 SEQ ID NO: 15 | 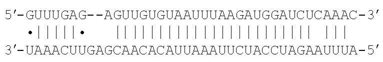 |

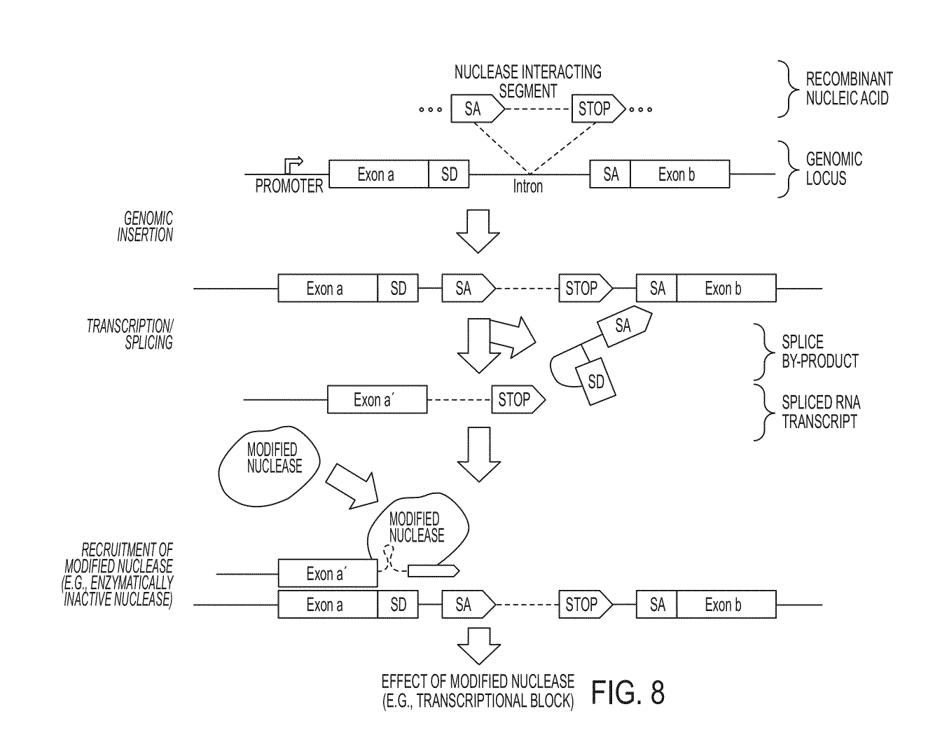

Further examples of base-paired structures that can be formed by a nuclease interacting segment and that interact with RNA-guided nucleases are disclosed in International Patent Application Publication Number WO/2013/176772, which published on Nov. 28, 2013, and is entitled, “METHODS AND COMPOSITIONS FOR RNA-DIRECTED TARGET DNA MODIFICATION AND FOR RNA-DIRECTED MODULATION OF TRANSCRIPTION,” the contents of which relating to base-paired structures (including, e.g., those depicted in FIG. 8 of the publication) are incorporated herein by reference in its entirety.

In some embodiments, a loop connects strands of the stem portion of a nuclease interacting segment. In some embodiments, a 4 base loop is included. However, it should be appreciated that other size loops can be included (e.g., 2, 3, 5, 6, 7, 8, 9, 10, or more). In some embodiments, the loop has the following sequence 5′-GAAA-3′. However, it should be appreciated that other sequences can be used for the loop as aspects of the disclosure are not limited in this respect.

In some embodiments, a nuclease interacting segment may include 5 to 35 of the 5′ bases (upper strand) and 5 to 35 of the 3′ bases (lower strand) of a based-paired stem shown in Table 1, wherein the stems are connected by a loop (e.g., a 5′-GAAA-3′ loop) to form an RNA segment. In some embodiment, a nuclease interacting segment may include 10 to 25 of the 5′ bases (upper strand) and 10 to 25 of the 3′ bases (lower strand) of a based-paired stem shown in Table 1, wherein the stems are connected by a loop (e.g., a 5′-GAAA-3′ loop) to form an RNA segment. In some embodiment, a nuclease interacting segment may include 15 to 20 of the 5′ bases (upper strand) and 15 to 20 of the 3′ bases (lower strand) of a based-paired stem shown in Table 1, wherein the stems are connected by a loop (e.g., a 5′-GAAA-3′ loop) to form an RNA segment.

A non limiting example of portions of base-paired structures from Table 1 that can be used to form a nuclease interacting segment includes 18 of the 5′ bases (upper strand) and 18 of 3′ bases (lower strand) of the based-paired stem from N. meningitidis shown in Table 1, wherein the stems are connected by the 5′-GAAA-3′ loop to form an RNA segment having the following sequence:

5'-GUUGUAGCUCCCUUUCUCGAAAGAGAACCGUUGCUACAAU-3'
(SEQ ID NO: 2, the loop is underlined).

Similarly, portions of the S. pyogenes stems shown in Table 1 can be connected by a loop (e.g., a 5′-GAAA-3′ loop) to form a nuclease interacting segment. A non-limiting example has the following sequence:

5'-GUUUUAGAGCUAGAAAUAGCAAGUUAAAAU-3'
(SEQ ID NO: 1, the loop is underlined).

However, it should be appreciated that other stem loop structures having other sequences capable of interacting with a nuclease can be used as described herein.

In some embodiments, a tail portion is included immediately 3′ of the downstream stretch of the nuclease interacting region. In some embodiments, the tail portion has a sequence that does not promote the formation of a stem-loop structure. In some embodiments, the tail portion is at least 5 nucleotides long (e.g., 5-10, 10-15, 15-20 nucleotides long). However, it should be appreciated that shorter or longer tail portions can be included. Moreover, in some embodiments, a tail portion is provided having a sequence that does promote formation of a stem-loop structure.

In some embodiments, a tail portion is included immediately 3′ of the downstream stretch of the nuclease interacting region that promotes stability of the RNA molecule (e.g., in vivo stability).

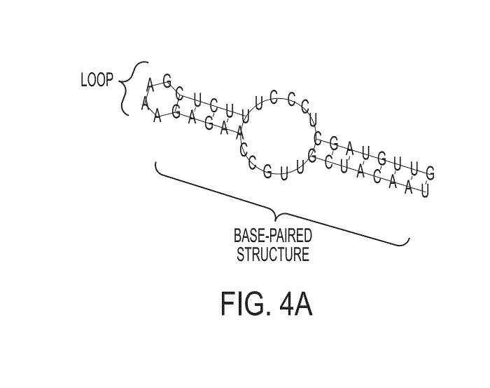

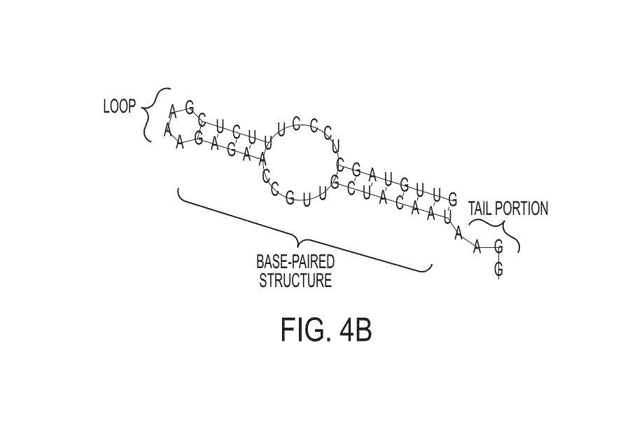

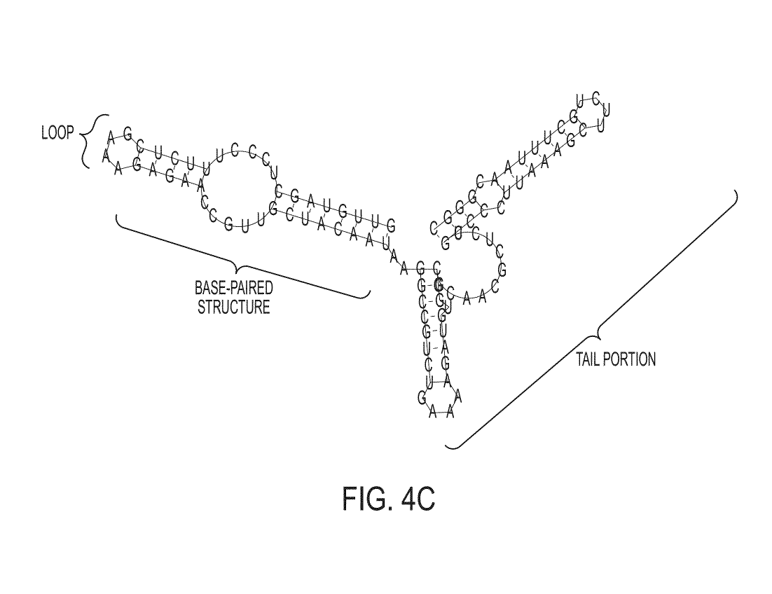

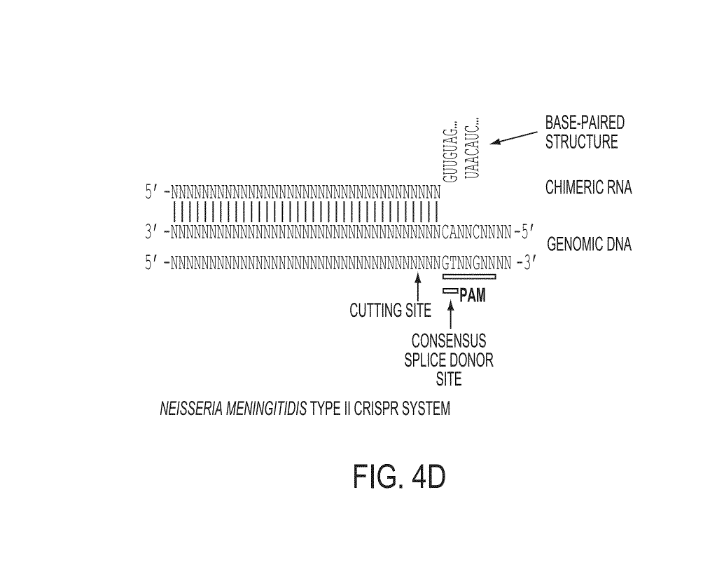

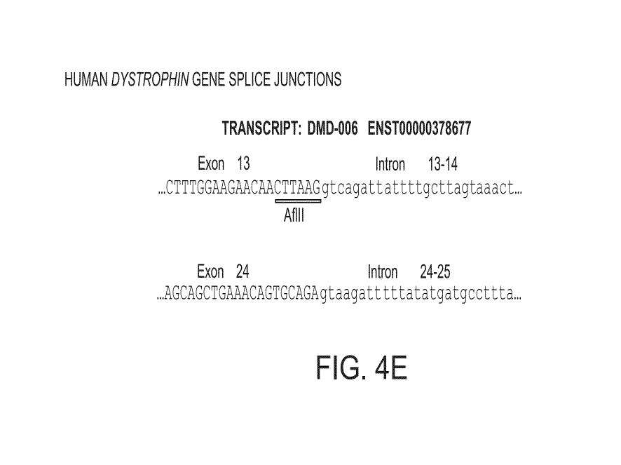

FIG. 4A illustrates a non-limiting embodiment of a nuclease interacting segment that comprises a RNA-guided nuclease interacting region that is a base-paired region that interacts with a CRISPR-associated nuclease from N. meningitides. The base-paired structure comprises i) a first strand having a sequence set forth as 5′ GUUGUAGCUCCCUUUCUC 3′ (SEQ ID NO: 16) that corresponds to the sequence of a targeting crRNA from N. meningitides and ii) a second strand having a sequence set forth as 5′ GAGAACCGUUGCUACAAU 3′ (SEQ ID NO: 17) that corresponds to the sequence of activating tracrRNA, in which the first and second strands are joined by a loop having a sequence set forth as 5′ GAAA 3′. FIGS. 4B and 4C illustrate non-limiting embodiments of nuclease interacting segments that comprise tail portions of different lengths, each tail portion corresponding to a 3′ sequence of an activating tracrRNA molecule from N. meningitides. The tail portion depicted in FIG. 4C comprises sequences capable of forming stem loop structures.

As illustrated in FIG. 4D, Cas9 nuclease from Neisseria meningitidis preferentially cuts within the portion of the genomic locus that is hybridized to the complementary targeting segment of the chimeric spliced RNA molecule, several bases (3-4 bases) immediately upstream from a 5′ GTNNGNN 3′ motif that is not hybridized to the targeting RNA segment.

FIG. 4E illustrates a non-limiting embodiment of a gene (the human Dystrophin gene) that contains a plurality of introns, some of which contain a preferred nuclease cleavage site for a Cas9 nuclease from Neisseria meningitidis. FIG. 4E illustrates two exon/intron boundaries of the human Dystrophin gene that will generate a non-hybridized 5′ GTNNGNN 3′ motif immediately downstream from a genomic exon that will hybridize to a targeting segment of a chimeric spliced RNA that would result from integration (followed by transcription and splicing) of a recombinant nucleic acid described herein into the illustrated intron. For example, integration of a recombinant nucleic acid described herein into Intron 13-14 (or Intron 24-25) will result in a chimeric spliced RNA molecule that includes Exon 13 RNA (or Exon 24) as the targeting segment followed by the nuclease interacting segment. When the chimeric spliced RNA binds to the complementary strand of genomic Exon 13 (or Exon 24), the genomic sequence that is immediately downstream from Exon 13 (or Exon 24), and that is not complementary or hybridized to the targeting segment of the chimeric spliced RNA, corresponds to the 5′ GTNNGNN 3′ motif (5′ GTCAGAT 3′ for Intron 13-14, and 5′ GTAAGAT 3′ for Intron 24-25). However, it should be appreciated that other sequences can support cleavage (e.g., even if they do not correspond exactly to the cleavage motif) even if the cleavage is not always as efficient, as aspects of the disclosure are not limited in this respect.

In some embodiments, a transcriptional terminator can be encoded downstream of the tail portion. In some embodiments, the transcriptional terminator includes a sequence that promotes the formation of a stem-loop structure. In some embodiments, a polyadenylation signal is encoded downstream of the nuclease interacting segment. In some embodiments, the polyadenylation signal is recognized by one or more factors (e.g., enzymes, co-factors) that cleave the 3′ portion of RNA encoded by the recombinant nucleic acid and polyadenylate the end produced by this cleavage. In some embodiments, the polyadenylation signal comprises the nucleotide sequence: AAUAAA. In some embodiments, the polyadenylation signal is a SV40 early, SV40 late , or BGH polyadenylation signal.

### RNA-Guided Nucleases:

In some embodiments, an RNA-guided nuclease is a CRISPR-associated nuclease. In some embodiments, Cas9 nucleases from one or more of the following organisms can be used N. meningitides, S. thermophiles, or T. denticola. Cas9 nucleases of orthologues of N. meningitides, S. thermophiles, or T. denticola may also be used. Further non-limiting examples of CRISPR-associated nucleases that may be used include those disclosed in International Patent Application Publication Number WO/2013/176772, which published on Nov. 28, 2013, and is entitled, “METHODS AND COMPOSITIONS FOR RNA-DIRECTED TARGET DNA MODIFICATION AND FOR RNA-DIRECTED MODULATION OF TRANSCRIPTION,” the contents of which relating to RNA-guided nucleases are incorporated herein by reference in its entirety.

As described herein, different nucleases show different relative preferences for different interacting segments of guide RNAs and different target sequences. In some embodiments, an interacting segment of a guide RNA binds to a nuclease, which then becomes activated and specific to a genomic sequence complementary to the guide portion of the RNA. The guide 0portion of the RNA is typically 20 nucleotides in length. However, in some embodiments, the guide portion may be 10, 11, 12, 13, 14, 15, 16, 17, 18, 19, 20, 21, 22, 23, 24, 25, 26, 27, 28, 29, 30 or more nucleotides in length. In some embodiments, the guide portion is in a range of 5 to 25, 10 to 30, 15 to 25, or 18 to 22 nucleotides in length.

In some embodiments, genomic target sequences complementary to a guide RNA have a protospacer adjacent motif (PAM) adjacent to their 3′ end. In some embodiments, the PAM sequence aids the nuclease in discriminating genomic targets for degradation. In aspects of the disclosure, nucleases are targeted to genomic sites by guide sequences (of a chimeric spliced RNA described herein) complementary to an exon at a position 5′ to a splice donor site. In such embodiments, if a sequence comprising the donor site is a PAM sequence recognized by the targeted nuclease, then the nuclease will cleave the genomic site within the exon. Accordingly, in some embodiments, nucleases are selected that are active against genomic targets with PAM sequences that contain splice donor sites (e.g., the PAM sequence, NNNNGTNN, which is recognized by the Cas9 enzyme of N. meningitides).

Table 2 below list different PAM sequences that are recognized by Cas9 nucleases of different organisms.

TABLE 2
PAM Sequences recognized by different
Cas9 nucleases

| N. | S. | T. | S. |
|---|---|---|---|
| menin- | thermo- | | |
| gitides | philus | denticola | pyogenes |
| NNNNGANN | NNAGAA | NAAAAN | NGG |
| NNNNGTTN | NNAGGA | NAAANC | GAG |
| NNNNGNNT | NNGGAA | NANAAC | NNGGN |
| NNNNGTNN | NNANAA | NNAAAC | |
| NNNNGNTN | NNGGGA | | |

N = A, G, T, or C

In some embodiments, a PAM sequence recognized by a particular nuclease (e.g., a PAM sequence recognized by a native nuclease of S. pyogenes) may not conform to a certain consensus sequence splice sequence. However, enzymes recognizing such sequences may be useful in certain contexts, e.g., in certain cells types where the PAM sequence comprises a sequence that is operative as a splice site.

### Splice Acceptor Sites:

In some embodiments, recombinant nucleic acids are provided that encode RNAs that have splice acceptor sites 5′ to a nuclease interacting region. In some embodiments, the recombinant nucleic acids insert within the intron of a genomic site that is transcribed in a cell. The resulting transcript is spliced between an endogenous splice donor site and the splice acceptor of the recombinant nucleic acid resulting in a chimeric guide RNA that comprises an upstream exon sequence fused to a nuclease interacting region and that targets a RNA-guided nuclease to the genomic site encoding the exon.

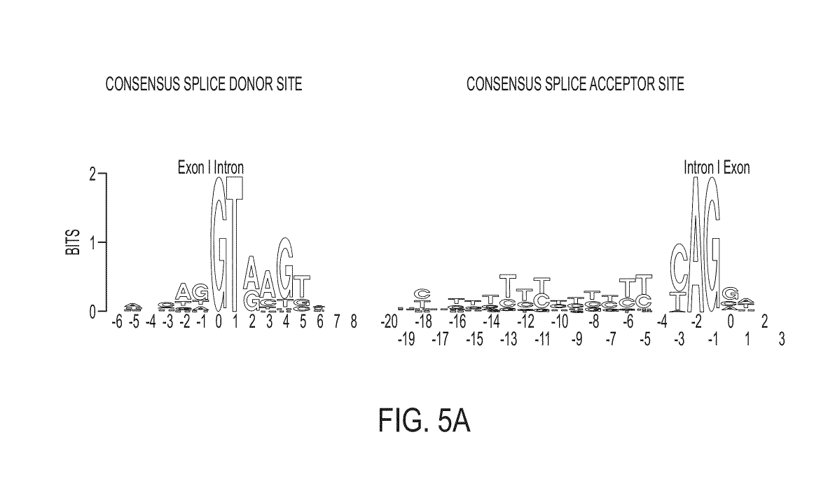

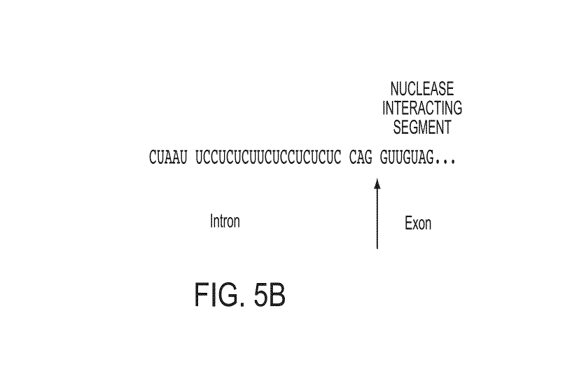

Thus, aspects of the disclosure utilize RNA splicing to remove introns from chimeric RNA transcripts to generate guide RNAs that target nucleases to particular genomic site. Each intron comprises a splice donor site at its 5′ end and an splice acceptor site at its 3′ end. FIG. 5A depicts a non-limiting embodiment of a consensus sequence of a splice donor site that has the sequence GU (encoded by GT) at the 5′ end of an intron. However, in some embodiments, a splice donor site may have the sequence AU (encoded by AT) or the sequence GC (encoded by GC) at the 5′ end of an intron.

FIG. 5A also depicts a non-limiting embodiment of a consensus sequence of a splice acceptor site that has a sequence AG at the 3′ end of an intron. However, in some embodiments, an acceptor site may have the sequence AC at the 3′ end of the intron.

In some embodiments, splice donor and acceptor site pairs are provided that contain GT and AG, respectively. In some embodiments, splice donor and acceptor site pairs are provided that contain AT and AC, respectively. In some embodiments, splice donor and acceptor site pairs are provided that contain GC and AG, respectively. In such embodiments, the splice acceptor site is generally provided on a recombinant nucleic acid construct, and the splice donor site is a natural site in the genome (as opposed to being provided recombinantly).

FIG. 5B depicts a non-limiting embodiment of a portion of a chimeric RNA having a splice acceptor site at the 3′ end of an intron linked at its 3′ end to a RNA interacting segment, which interacts with a nuclease.

### Modified RNA-Guided Nuclease:

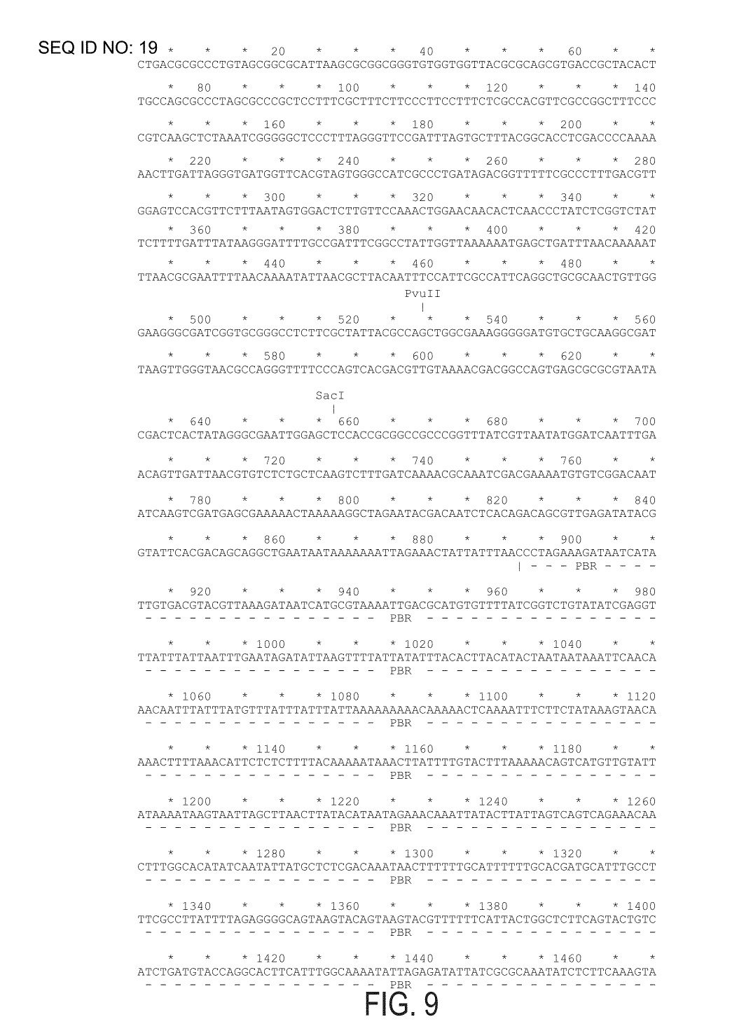

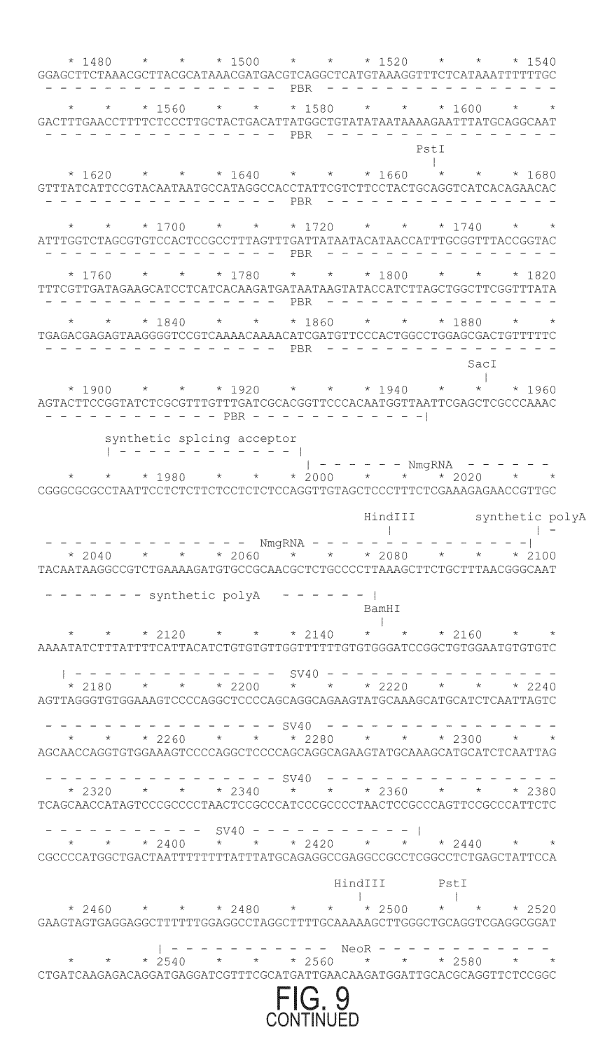

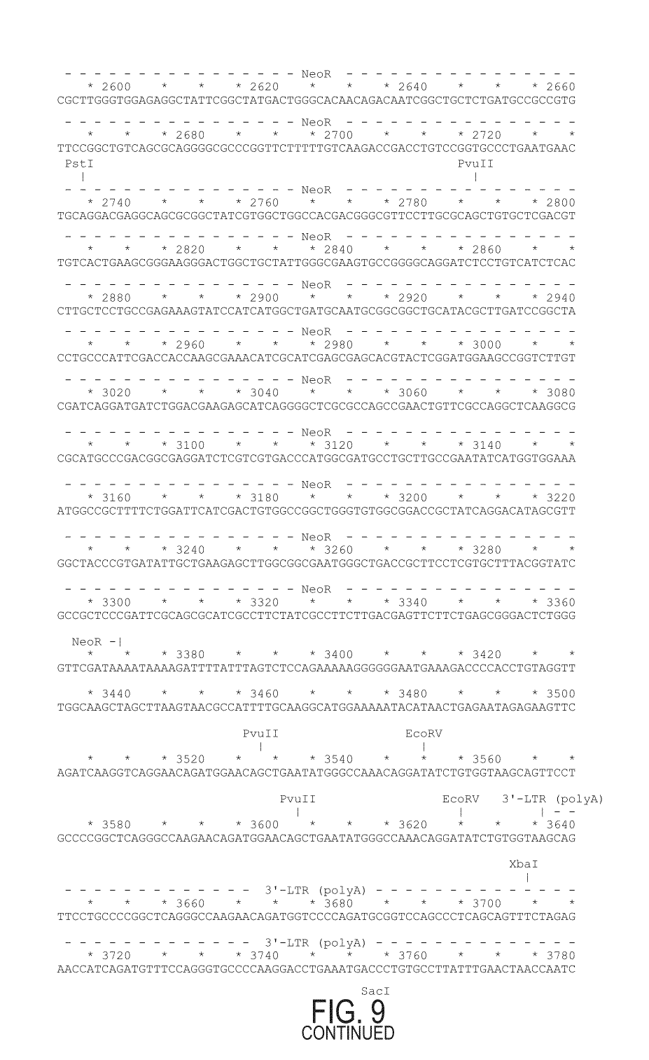

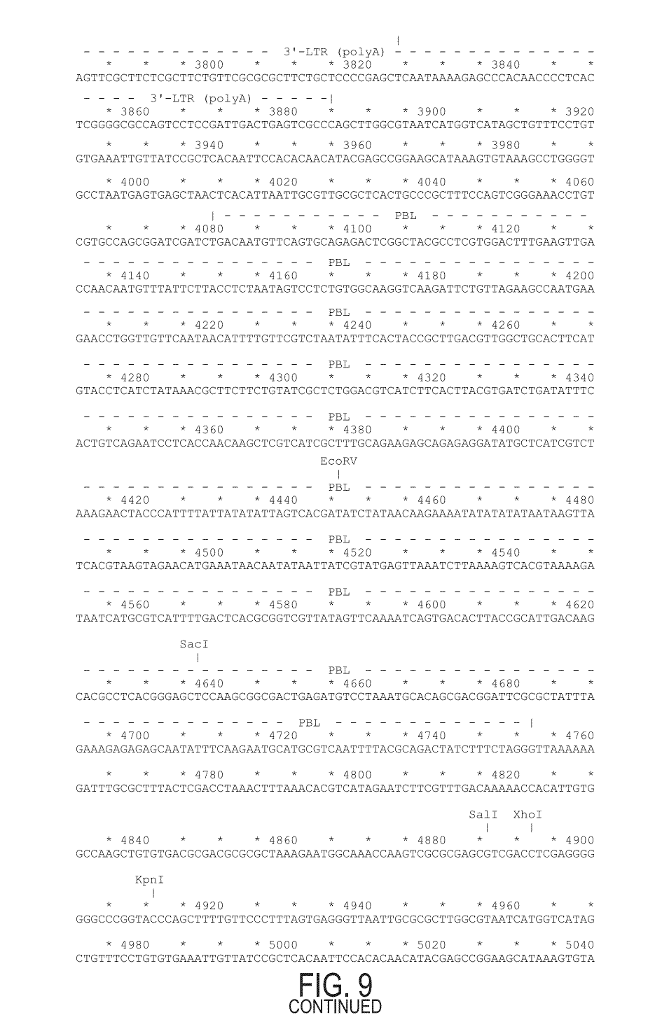

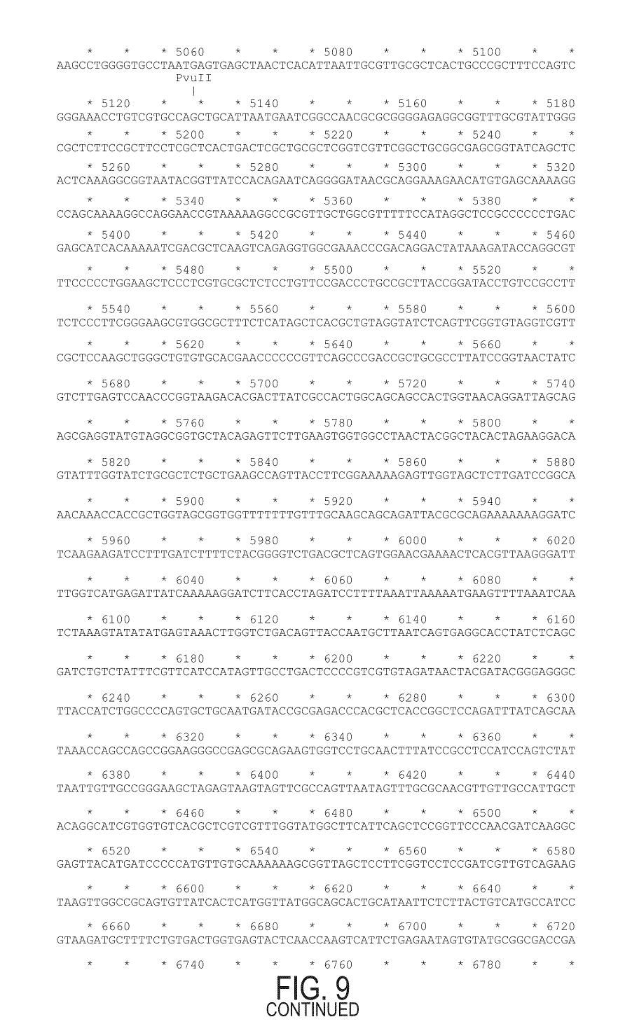

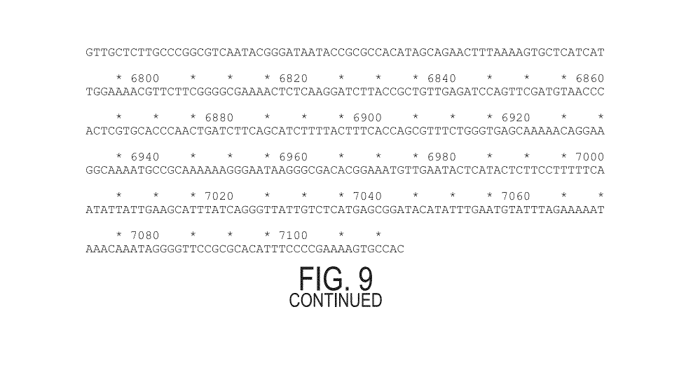

In some embodiments, a modified nuclease can be guided to a genomic target site by a chimeric spliced RNA molecule described herein. In some embodiments, the modified nuclease can be enzymatically inactive (e.g., it does not cleave DNA). In some embodiments, an enzymatically inactive nuclease binds to a chimeric spliced RNA molecule associated with a genomic locus for an exon (e.g., the exon that is included in the chimeric spliced RNA molecule) and can act as a transcriptional block to prevent or reduce the efficiency of transcription past the site at which the modified nuclease is bound. FIG. 9 illustrates a non-limiting embodiment of a system described herein wherein the recombinant nucleic acid integration, transcription, and splicing are identical to those illustrated in FIG. 1. However, the nuclease that is present in the cell is a modified nuclease that binds to the chimeric spliced RNA but does not cleave the associated genomic sequence.

It should be appreciated that a modified nuclease that is capable of binding and preventing transcription or reducing transcriptional efficiency can act on both alleles of a genetic locus (or at multiple alleles of a genetic locus) in a cell. Accordingly, methods and compositions described herein can be used to silence one or more alleles of a genetic locus in a cell.

In some embodiments, a library of host cells having insertional constructs integrated into different genomic loci (e.g., into introns of different genes, and/or into different introns of one or more genes) can be created. Different host cells in the library can have one or more silenced genetic loci (e.g., 2, 3, 4, 5, or more) depending on the number and location of independent integration events within each host cell. In some embodiments, a library of host cells described herein can be screened to identify one or more genetic loci associated with a phenotype of interest (e.g., a response or susceptibility to one or more therapeutic compounds).

In some embodiments, a modified nuclease can have one or more novel functions in addition to, or instead of, being enzymatically inactive. In some embodiments, a nuclease can be modified to include a detectable moiety. In some embodiments, a nuclease can be modified to include an additional peptide segment. An additional peptide segment can be attached at the N-terminus, C-terminus, and/or between the N-terminal and C-terminal positions of the nuclease. In some embodiments, the additional peptide segment is a domain that has an effector function. In some embodiments, the additional peptide segment includes a linker peptide. In some embodiments, the effector function is an enzymatic function and/or a regulatory function. Non-limiting examples of effector functions include: transcriptional enhancement, transcriptional repression, methylation (e.g., methylation of DNA and/or DNA-associated proteins), demethylation (e.g., demethylation of DNA and/or DNA-associated proteins), other DNA or RNA modification activities, binding to one or more regulatory proteins, and/or other functions as aspects of the disclosure are not limited in this respect.

Accordingly, methods and compositions described herein also can be used to produce a library of host cells, each having a modified nuclease with an effector function that is targeted to a different genetic locus (e.g., introns ofdifferent genes and/or different introns of one or more genes). It should be appreciated that these host cells can be screened as described herein to identify one or more cells having a property of interest.

In some embodiments, compositions and methods described herein can be used to introduce modifications (e.g., mutations) at one or more loci (e.g., at one or more alleles of one or more loci as described herein) in a single cell or in a plurality of cells (for example in a cell culture). In some embodiments, a modified cell (for example an embryonic or other stem cell that is modified as described herein) can be used to generate a multicellular organism that has the modification (for example one or more mutations) of the original cell.

In some embodiments, compositions or methods described herein can be used to modify one or more cells in a multicellular organism. In some embodiments, a composition described herein can be introduced (e.g., by injection or other technique) into an embryo (or other multicellular developmental stage of a multicellular organism, for example a blastocyst). This can result in modification of one or more cells (e.g., all cells) to produce an adult multicellular organism for which all cells or a subset of cells are modified (e.g., the multicellular organism is chimeric for one or more modifications at one or more genetic loci). It should be appreciated that in this embodiment different cells in a multicellular organism may have different modifications since different modifications are likely to have been introduced into the different cells in the early developmental stage.

In some embodiments, compositions and methods described herein can be used to modify one or more cells of a juvenile or adult multicellular organism. For example, a composition described herein can be introduced (e.g., by injection or other technique) at one or more locations in a juvenile or adult multicellular organism. At each location, one or more cells may be modified as described herein.

Non-limiting examples of multicellular organisms include mammals, birds, reptiles. Non-limiting examples of mammals include humans, mice, rabbits, rats, sheep, goats, cows, and horses.

Exemplary embodiments of the invention will be described in more detail by the following examples. These embodiments are exemplary of the invention, which one skilled in art will recognize is not limited to the exemplary embodiments.

### EXAMPLES

### Example 1

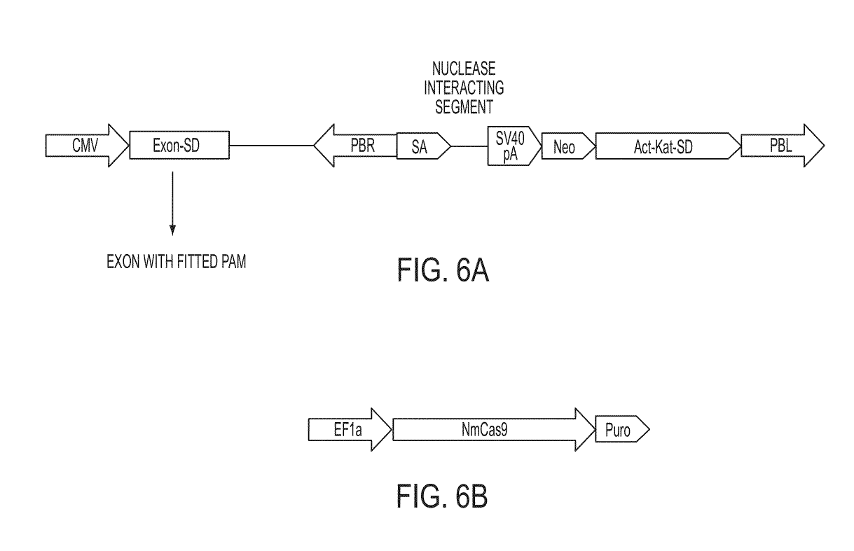

FIG. 6 illustrates a non-limiting embodiment of an experimental system for generating a chimeric spliced RNA that includes i) an RNA targeting segment corresponding to an exon spliced to ii) a nuclease interacting segment. The nucleic acid construct illustrated in FIG. 6A includes a promoter (CMV promoter) that can drive transcription of an RNA molecule containing i) an experimental target segment (Exon) immediately upstream of ii) a splice donor site (SD) followed by iii) an intervening segment (containing a transposon repeat—PBR) upstream of iv) a splice acceptor site (SA) that is upstream of v) a nuclease interacting segment followed by vi) a polyadenylation site (SV40 pA). In some embodiments, the nucleic acid construct may contain one or more additional elements, including, without limitation, sequences encoding tags (e.g., a MYC epitope) or labels, sequences encoding proteins, (e.g., fluorescent proteins), sequences encoding an internal ribosomal entry site (IRES) that is configured to express one or more proteins from a transcript encoded by the nucleic acid, etc. After this transcribed RNA molecule is spliced, the resulting chimeric spliced RNA contains the Exon spliced to the nuclease interacting segment (the splice donor and splice acceptor sites are spliced out along with the intervening RNA segment). The ability of this chimeric spliced RNA to target a DNA molecule containing the Exon (e.g., followed by the splice donor site in the context of an appropriate cleavage site) can be evaluated using an appropriate assay. In some embodiments, an assay can include using a Cas9 nuclease to determine whether the chimeric spliced RNA can promote cleavage of the DNA molecule containing the Exon. In some embodiments, the assay can be performed in a cell that includes both the test construct of FIG. 6A (for example on an independently replicating vector or integrated into a genomic locus) and a construct that expresses a Cas9 nuclease. FIG. 6B illustrates a non-limiting embodiment of a construct that can express a Neisseria meningitidis Cas9 nuclease. The construct of FIG. 6B also can be on an independently replicating vector integrated into a genomic locus.

It should be appreciated that one or more selectable markers can be used to select for the presence of the constructs of FIG. 6A and FIG. 6B in host cells of interest. The markers shown in FIG. 6A and FIG. 6B are Neomycin (Neo) and Puromycin (Puro) resistance markers, respectively. However, it should be appreciated that other selectable markers can be used as aspects of the disclosure are not limited in this respect.

It should be appreciated that constructs such as illustrated in FIG. 6A can be used to evaluate the effectiveness of different target sequences, different cleavage sequences, different nuclease interacting sequences, and/or other factors that can be varied.

### Example 2

In some embodiments, the construct illustrated in FIG. 6A can be used to integrate the segment that is between the transposon ends (PBR and PBL) into a genomic locus (e.g., into an intron) in order to evaluate the ability of the nuclease interacting segment to be spliced to the 3′ end of a natural exon transcribed from a genomic locus. The genomic integration of the segment between the transposon ends can be promoted by a transposase (e.g., PBase). It should be appreciated that this results in a different use of the construct of FIG. 6A than described in Example 1. In Example 1, the splicing occurs with the experimental exon (Exon) that is transcribed from the CMV promoter on the construct. In contrast, after integration into a genomic intron, the splicing occurs with a natural exon that is transcribed from a genomic locus. Accordingly, it should be appreciated that the CMB Exon-SD portion is not required for integration.

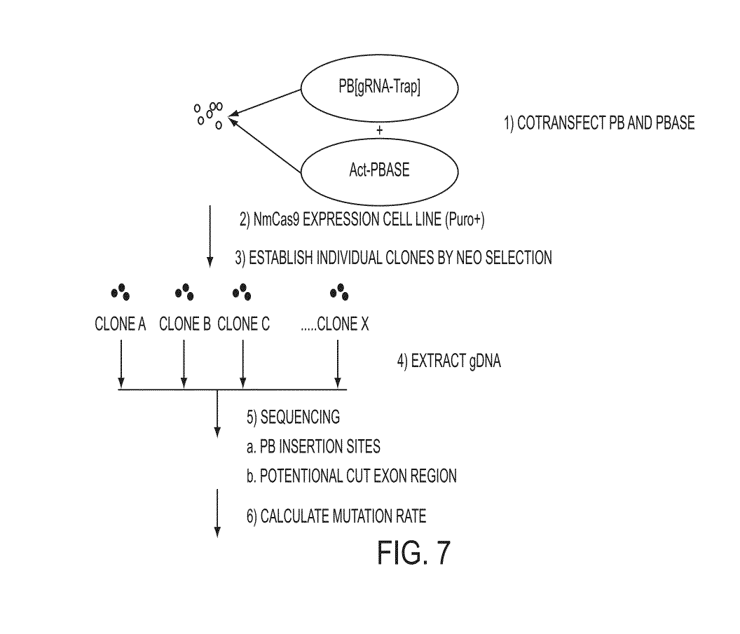

FIG. 7 illustrates a non-limiting embodiment of an experimental outline for evaluating the effectiveness of a system described herein for producing mutations at one or more genomic loci in a host cell. In 1), a construct such as the one illustrated in FIG. 6A (e.g., the segment between and including PBR and PBL) is cotransfected along with a transposase (PBase) into a host cell to promote integration into a genomic locus of a host cell. In 2), a host cell expressing Cas9 from Neisseria meningitides (NMCas9) from a construct that also encodes a selectable marker (Puro) can be used. In 3), a plurality of different individual host cell clones that each contain an integrated transposon segment (the segment between the transposon repeats of FIG. 6A) can be selected for using a selectable marker that is encoded on the transposon segment (Neo). In 4), genomic DNA (gDNA) from the different host cell clones can be extracted. In 5), the gDNA can be sequenced to identify a) the different insertion sites (PB insertion sites) in the different host cell clones, and b) potential cut sites in exons immediately upstream from the insertion sites. In 6), mutation rates (e.g., caused by cleavage and error-associated repair of the cut sites) can be calculated by determining the frequency at which errors are found at potential cut sites. It should be appreciated that mutation rates at two or more alleles of a genomic locus can be determined.

It should be appreciated that in some embodiments, the transposon segment can be excised (e.g., after a mutation is introduced at an exon) by the further action of a transposase (e.g., PBase). In some embodiments, cells from which the transposon segment has been excised can be identified by having a further marker encoded on the transposon segment such as the Kat marker illustrated in FIG. 6A. Kat refers to the Katushka red fluorescent protein and is regulated by the actin promoter. It should be appreciated that, in some embodiments, a Kat transcript is relatively unstable in cells as it lacks a polyadenylation tail. Thus, in some embodiments, stability of the transcript will increase when the nucleic acid encoding the transcript is inserted into an intron upstream of a polyadenylation site. This configuration facilitates identification of a cell that harbors a useful transposon insertion, by detecting expression of the fluorescent protein, which would be expressed above a detection threshold only in cells having stable polyadenylated transcripts. In some embodiments, detection of the marker may be used to identify and/or sort cells with transposon insertions into transcriptional units. Cells that are Kat free after further action of a transposase can be further evaluated (e.g., via sequencing) to confirm that the transposon segment has been excised. However, it should be appreciated that other markers or techniques can be used to identify cells from which a transposon segment has been removed as aspects of the disclosure are not limited in this respect.

### Example 3

FIG. 9 provides a non-limiting example of a sequence of an insertional recombinant nucleic acid. The recombinant nucleic acid comprises a splice acceptor site upstream of a nucleic acid region that encodes an RNA segment capable of interacting with a RNA-guided nuclease.

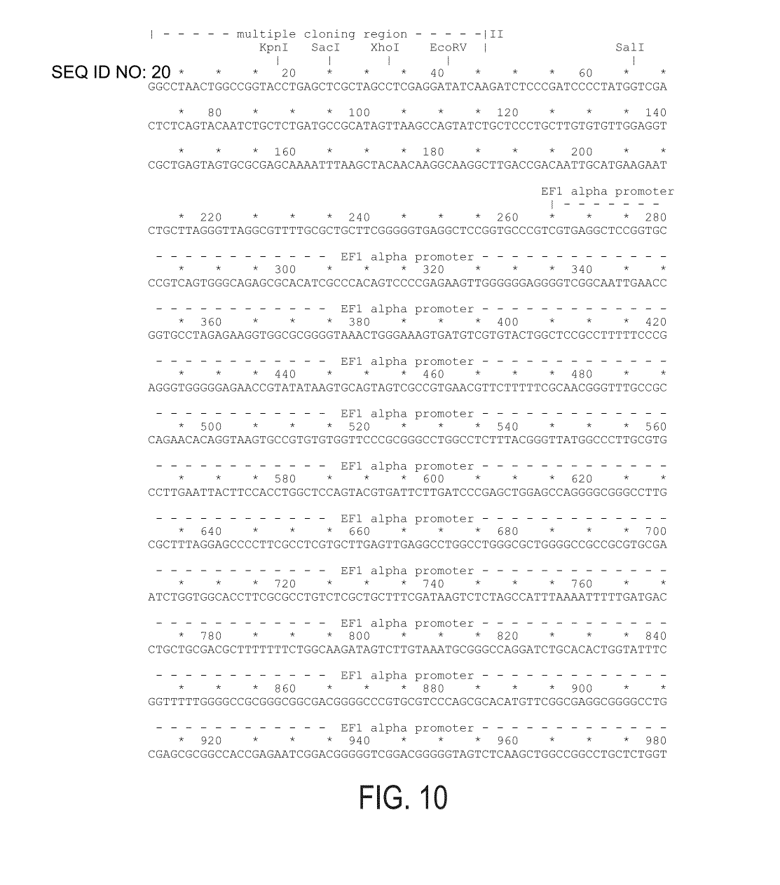

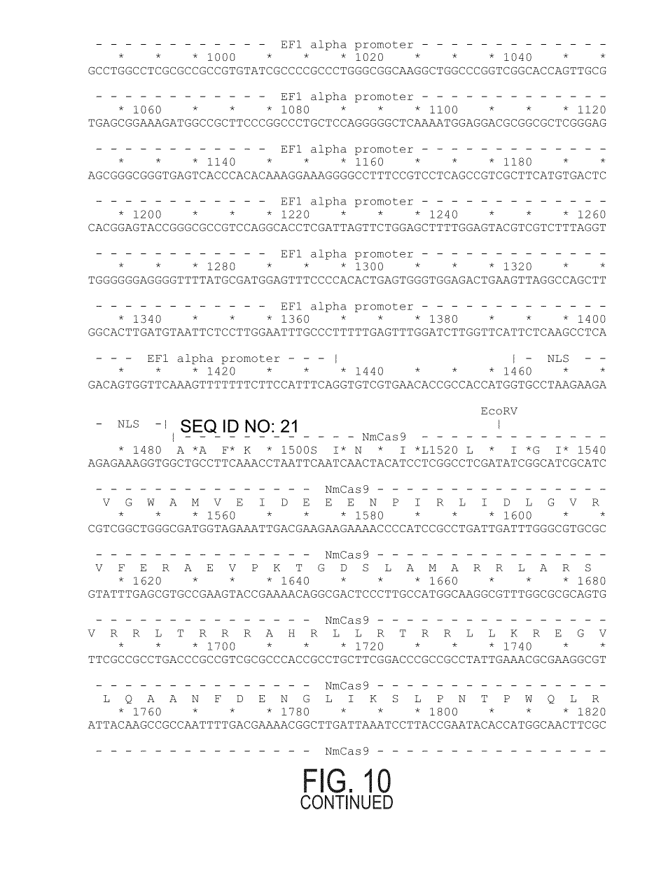

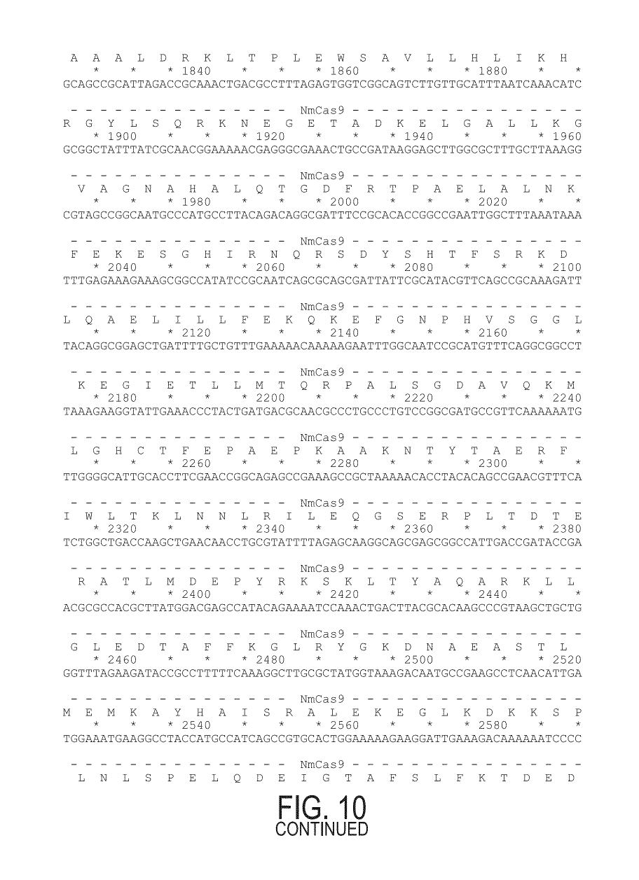

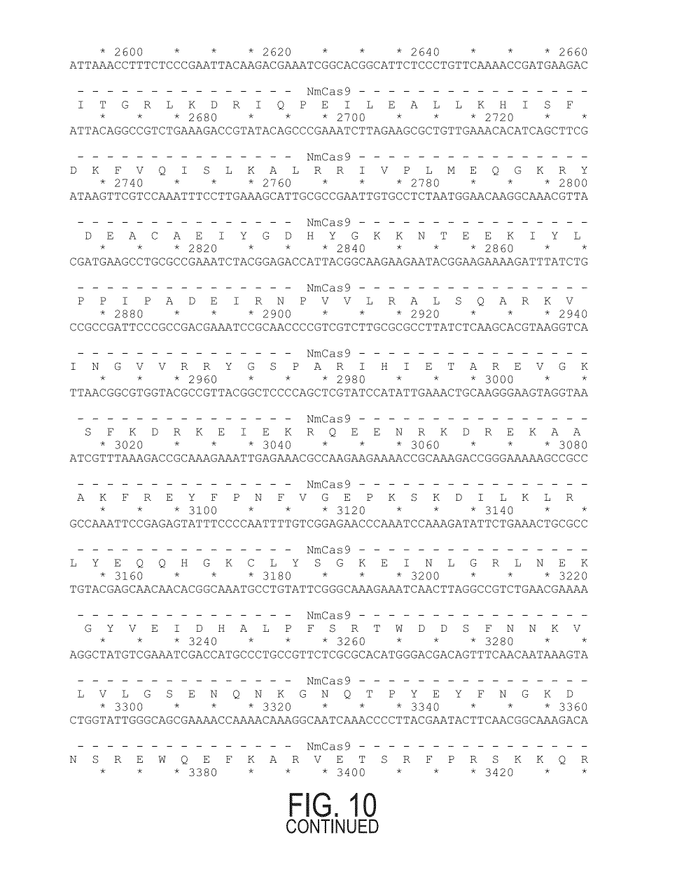

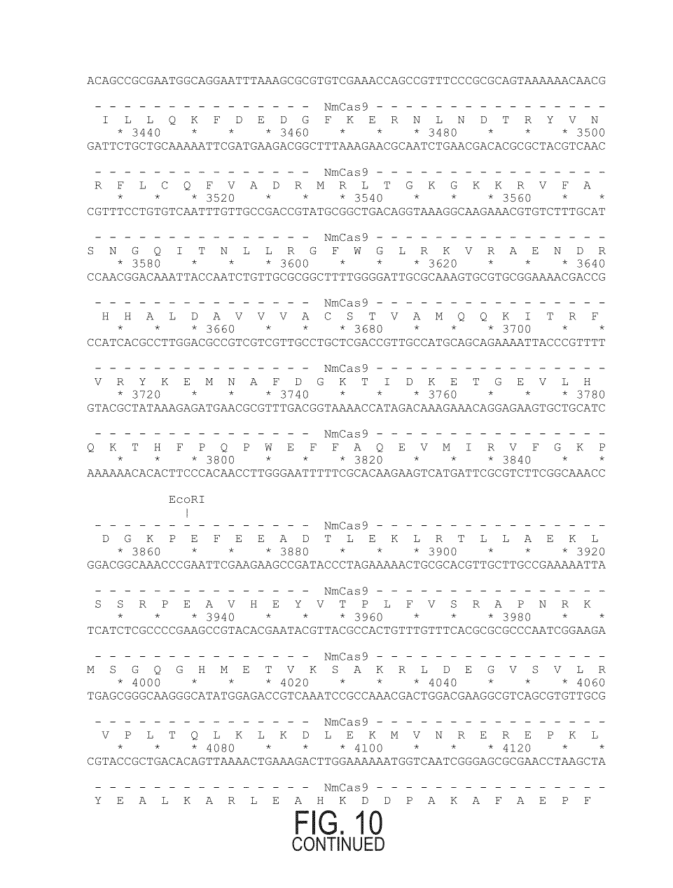

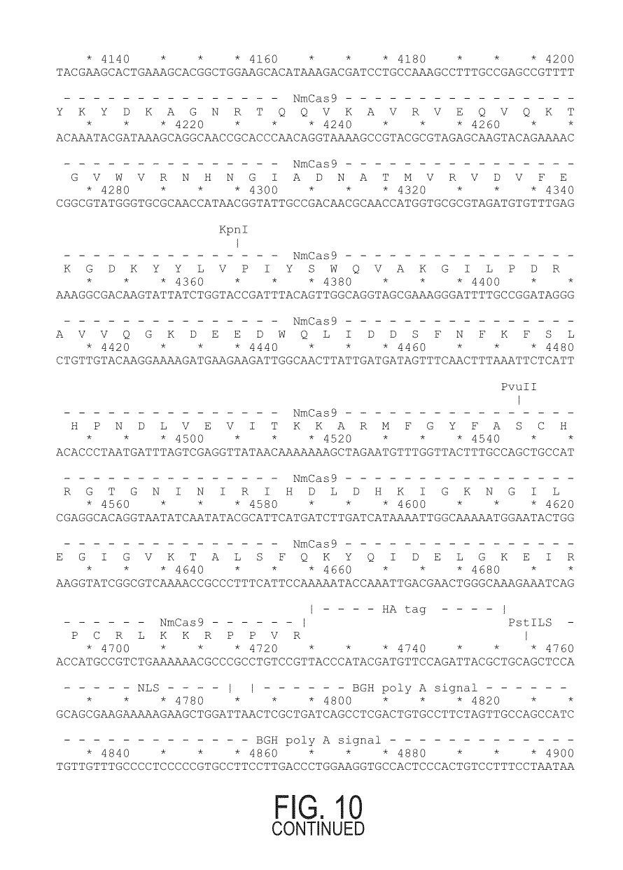

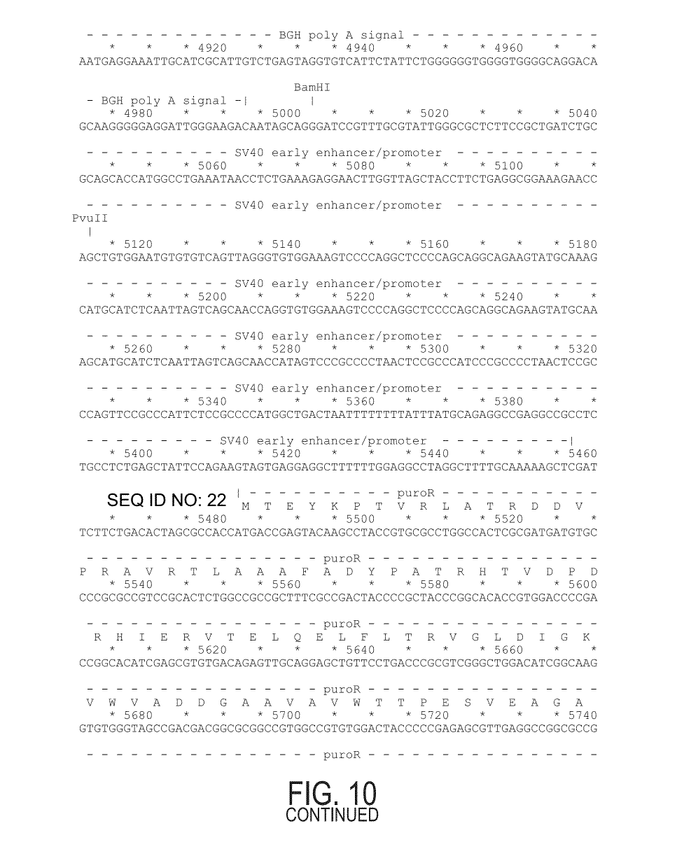

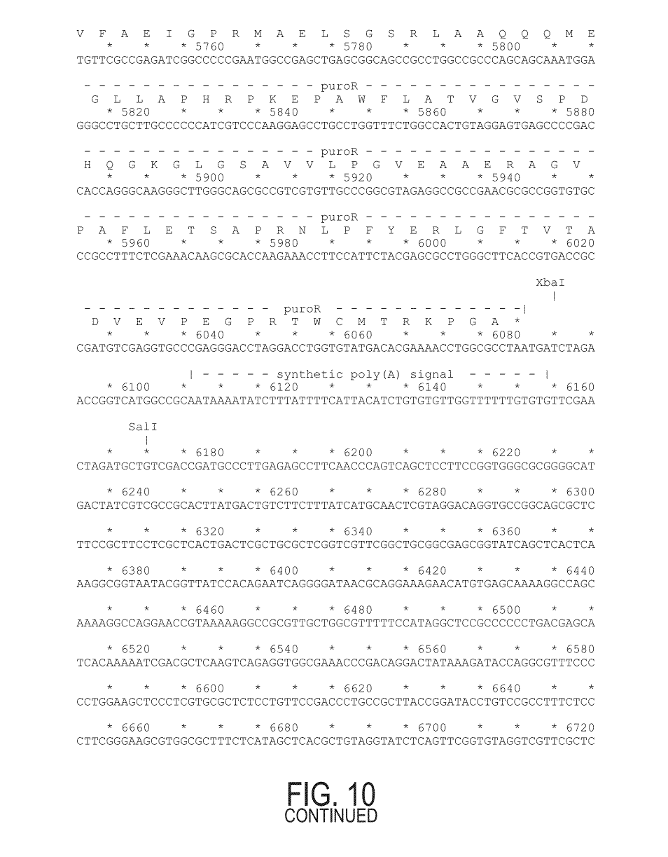

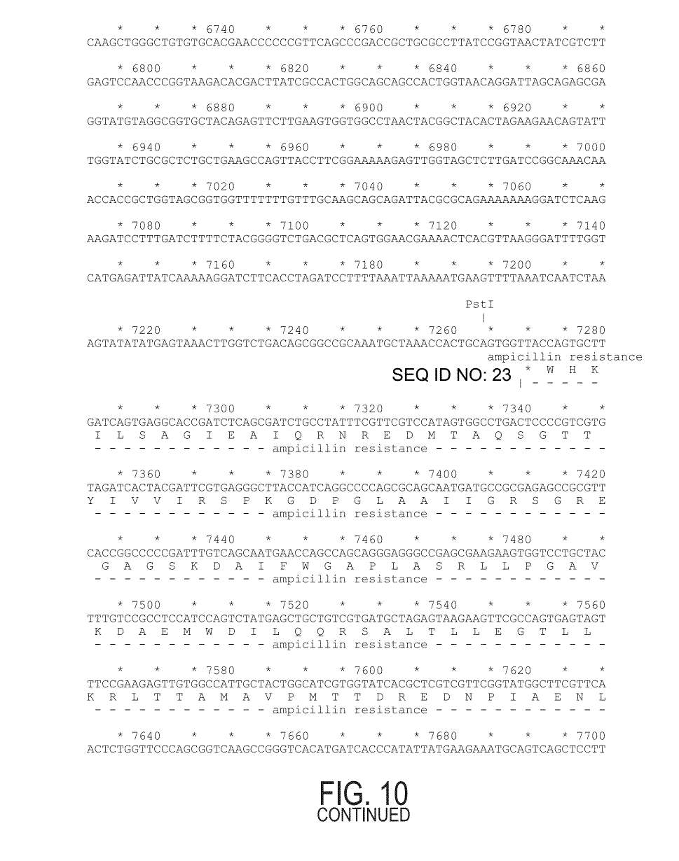

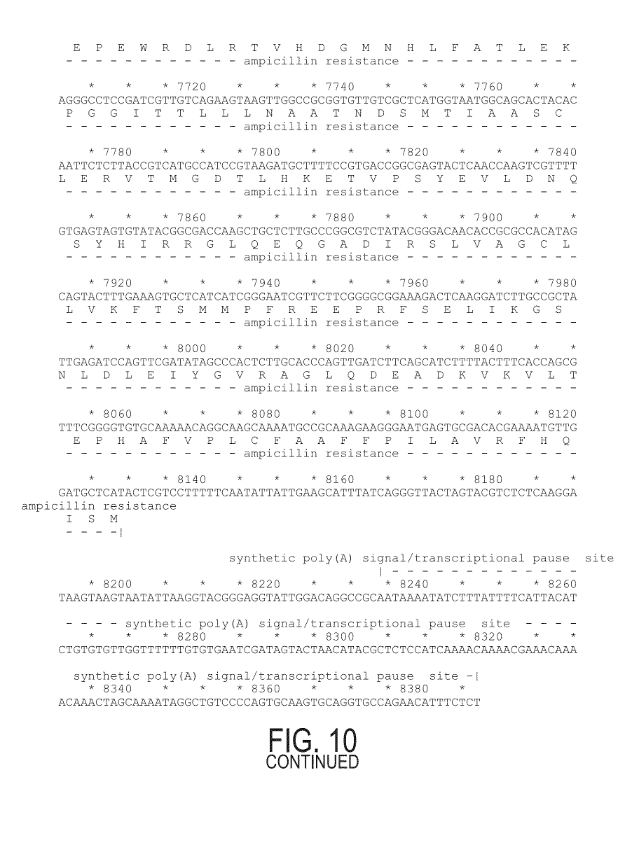

FIG. 10 provides a non-limiting example of a sequence of a nucleic acid engineered to express a Cas9 nuclease.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A method of producing, in a eukaryotic cell, a target-specific RNA molecule capable of guiding a DNA nuclease to a genomic target, the method comprising introducing a recombinant nucleic acid into a eukaryotic cell, wherein the recombinant nucleic acid comprises a first nucleic acid region that encodes a splice acceptor site upstream of a second nucleic acid region that encodes an RNA segment capable of interacting with an RNA-guided DNA nuclease.

2. A method of producing, in a eukaryotic cell, a target-specific RNA molecule capable of guiding a DNA nuclease to a genomic target, the method comprising integrating a recombinant nucleic acid into a genomic locus of a eukaryotic cell, wherein the recombinant nucleic acid comprises a first nucleic acid region that encodes a splice acceptor site upstream of a second nucleic acid region that encodes an RNA segment capable of interacting with an RNA-guided DNA nuclease.

3. A method of promoting RNA-guided cleavage of a genomic DNA within a cell, the method comprising:
producing, in a eukaryotic cell, an RNA molecule that comprises a first RNA segment spliced to a second RNA segment, wherein the first RNA segment comprises an exonic sequence transcribed from a genomic locus and the second RNA segment comprises an RNA segment capable of interacting with an RNA-guided DNA nuclease, and
expressing, in the eukaryotic cell, the RNA-guided DNA nuclease.

4. The method of claim 1, wherein the recombinant nucleic acid is a DNA molecule.

5. The method of claim 1, wherein the recombinant nucleic acid comprises transposon terminal sequences.

6. The method of claim 5, wherein the transposon terminal sequences comprise inverted terminal repeat sequences (ITRs).

7. The method of claim 5, wherein the transposon terminal sequences comprise direct terminal repeat sequences.

8. The method of claim 7, wherein the direct terminal repeat sequences flank the ITRs.

9. The method of claim 5, wherein the transposon terminal sequences comprise a 5′ terminal CCY and a 3′ terminal GGG.

10. The method of claim 9, wherein the transposon terminal sequences comprise a 5′ terminal CCC and a 3′ terminal GGG.

11. The method of claim 5, wherein the transposon terminal sequences target TTAA insertion sites.

12. The method of claim 5, wherein the transposon terminal sequences comprise PiggyBac transposon-specific inverted terminal repeat sequences (ITRs).

13. The method of claim 5, wherein the transposon terminal sequences comprise Tagalong transposon-specific inverted terminal repeat sequences (ITRs).

14. The method of claim 1, wherein recombinant nucleic acid further comprises a third nucleic acid region encoding a selection or screening marker.

15. The method of claim 14, wherein the selection or screening marker is an antibiotic resistance protein or a fluorescent or bioluminescent protein.

16. The method of claim 1, wherein the splice acceptor site comprises a sequence set forth as 5′-X₁X₂X₃-3′,
wherein:
X₁ is A,
X₂ is G or C, and
X₃ is A, G, C, or U, wherein a 3′ splice junction is between X₂ and X₃.

17. The method of claim 16, wherein X₂ is G.

18. The method of claim 16, wherein X₃ is A, G or C.

19. The method of claim 1, wherein the splice acceptor site comprises a sequence set forth as 5′-X₁X₂X₃X₄X₅-3′,
wherein:
X₁ is A, C or U,
X₂ is A,
X₃ is G,
X₄ is A, G or C, and
X₅ is A, U or C, wherein a 3′ splice junction is between X₃ and X₄.

20. The method of claim 1, wherein the splice acceptor site comprises a sequence set forth as 5′-X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂-3′ (SEQ ID NO: 18),
wherein:
X₁, X₃, X₅, X₇, X₉, X₁₂, X₁₅, X₁₆, and X₁₇ are each independently selected from A, G, C, and U,
X₂ is C or G,
X₄ is U,
X₆, X₈, X₁₀, X₁₁, X₁₃, X₁₄ are each independently selected from G, C, and U,
X₁₈ is A, C or U,
X₁₉ is A,
X₂₀ is G,
X₂₁ is A, C, or G, and
X₂₂ is A, U or C, wherein a 3′ splice site is between X₂₀ and X₂₁.

21. The method of claim 1, wherein the nuclease interacting segment comprises at least one stem portion that interacts with the RNA-guided DNA nuclease.

22. The method of claim 21, wherein the nuclease interacting segment comprises first and second stem portions that are separated by non-complementary RNA nucleotides.

23. The method of claim 21, wherein the first stem portion comprises a strand having a nucleotide sequence set forth as 5′-GUUGUAGC-3′.

24. The method of claim 21, wherein the second stem portion comprises a nucleotide sequence set forth as 5′-UUCUC-3′.

25. The method of claim 21, wherein complementary base pairs of the two strands of the second stem portion are covalently linked through a loop structure.

26. The method of claim 1, wherein the nuclease interacting segment comprises a sequence set forth as 5′-GUUUUAGAGCUAGAAAUAGCAAGUUAAAAU-3′ (SEQ ID NO:
1).

27. The method of claim 1, wherein the eukaryotic cell is a mammalian cell.

28. The method of claim 1, wherein the eukaryotic cell is a plant cell.

29. The method of claim 27, wherein the mammalian cell is a human cell.

30. The method of claim 1, wherein the recombinant nucleic acid encodes the RNA-guided DNA nuclease.

31. The method of claim 1, wherein the RNA-guided DNA nuclease is a CRISPR-associated (Cas) nuclease.

32. The method of claim 31, wherein the Cas nuclease is a Type II Cas nuclease.

33. The method of claim 32, wherein the Cas nuclease is a Cas9 nuclease.

34. The method of claim 33, where the Cas9 nuclease is a Neisseria meningitides Cas9 nuclease (NmCas9).

35. The method of claim 34, where the Cas9 nuclease is a Streptococcus thermophiles Cas9 nuclease.

36. The method of claim 1, wherein the RNA-guided DNA nuclease introduces single-stranded breaks in DNA.

37. The method of claim 1, wherein the RNA-guided DNA nuclease introduces double-stranded breaks in DNA.

38. The method of claim 3, wherein the RNA-guided DNA nuclease is expressed under conditions that promote i) interaction between the RNA-guided DNA nuclease and the second RNA segment of the RNA molecule, and ii) DNA cleavage at one or more genomic loci encoding the exonic sequence.

39. The method of claim 38, wherein the one or more genomic loci are two or more alleles encoding the exonic sequence.

40. The method of claim 39, wherein the two or more alleles are two alleles in a mammalian cell.

41. The method of claim 38, wherein DNA cleavage occurs within 5 base pairs upstream of a splice donor site of the exonic sequence.

42. A method of producing, in a eukaryotic cell, a target specific nucleic acid that guides a DNA modifying enzyme, the method comprising introducing a recombinant nucleic acid into a eukaryotic cell, wherein the recombinant nucleic acid comprises a first nucleic acid region that encodes a splice acceptor site upstream of a second nucleic acid region that encodes an RNA segment capable of interacting with the DNA modifying enzyme.

43. The method of claim 42, wherein the DNA modifying enzyme is an RNA-guided DNA nuclease.

44. The method of claim 1, wherein the eukaryotic cell is a stem cell.

45. A nucleic acid comprising a first nucleic acid region that encodes a splice acceptor site upstream of a second nucleic acid region that encodes an RNA segment capable of interacting with a DNA modifying enzyme.

46. The nucleic acid of claim 45, wherein the DNA modifying enzyme is an RNA-guided DNA nuclease.
